(19) United States
(12) Patent Application Publication
(10) Pub. No.: US 2023/0069191 A1
(43) Pub. Date: Mar. 2, 2023

(54) METHOD FOR QUANTITATING NUCLEIC ACID LIBRARY

(71) Applicant: EDGE BIOSYSTEMS, INC., San Jose, CA (US)

(72) Inventors: Richard Fekete, San Mateo, CA (US); Lei Xi, San Mateo, CA (US)

(21) Appl. No.: 17/796,558

(22) PCT Filed: Jan. 29, 2021
(86) PCT No.: PCT/US21/15859

§ 371 Date: Jul. 29, 2022

Related U.S. Application Data
(60) Provisional application No. 62/968,898, filed on Jan. 31, 2020.

(51) Int. Cl. C12Q 1/6823; C12N 15/10

(52) U.S. Cl. CPC C12Q 1/6823; C12N 15/1065; C12Q 2600/16; C12Q 1/6851

(57) ABSTRACT

A method for quantitating a plurality of nucleic acid molecules is provided. The method includes contacting a plurality of detectably-labeled probes and a plurality of extension primers with the plurality of nucleic acid molecules, each detectably-labeled probe including a first labeled nucleic acid domain having a first label; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating a number of the plurality of target nucleic acid fragments based on signals detected upon a single cycle of extension reactions.

### CROSS-REFERENCE TO RELATED APPLICATION

This application claims priority to U.S. Provisional Patent Application No. 62/968,898, filed Jan. 31, 2020, the contents of which are incorporated by reference in the entirety.

### REFERENCE TO A SEQUENCE LISTING

The instant application contains a Sequence Listing which has been submitted electronically in ASCII format and is hereby incorporated by reference in its entirety. Said ASCII copy, created on Jan. 29, 2021, is named BBQI000100PC_SL.txt and is 10,021 bytes in size. The information contained in this file is hereby incorporated by reference.

### BACKGROUND

Next-generation sequencing (NGS) technologies enable the high-throughput, massively parallel sequencing of nucleic acid molecules. NGS technologies can determine the nucleotide base sequence of millions of nucleic acid molecules (for example, RNA or DNA) in a sample. Moreover, NGS can determine the quantity of one or more nucleic acid sequences in a sample due to that the rate at which individual nucleic acid sequences are determined is correlated to the relative abundance of that individual nucleic acid sequence in the sample. Examples of NGS services and products include those provided by companies including Illumina, Oxford Nanopore, Pacific Biosciences, Ion Torrent, Roche 454 Pyrosequencing.

### SUMMARY

In one aspect, the present disclosure provides a method for quantitating a plurality of nucleic acid molecules comprising a plurality of target nucleic acid fragments, the method comprising contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating a number of the plurality of target nucleic acid fragments based on signals detected upon extension reactions.

Optionally, calculating the number of the plurality of target nucleic acid fragments is based on the first signal detected upon a single cycle of extension reactions.

Optionally, a total number of hydrolyzed detectably-labeled probes is substantially same as a total number of the plurality of target nucleic acid fragments prior to contacting the plurality of detectably-labeled probes and the plurality of extension primers with the plurality of nucleic acid molecules.

Optionally, subsequent to initially contacting the plurality of detectably-labeled probes and the plurality of extension primers with the plurality of nucleic acid molecules, and prior to detecting the first signal, no additional cycle of extension reactions is performed other than a single cycle of extension reactions.

Optionally, each of the plurality of target nucleic acid fragments comprises a first adapter sequence, a target insert sequence, and a second adapter sequence; the first adapter sequence is linked to the second adapter sequence through the target insert sequence; hydrolyzing each of the plurality of detectably-labeled probes comprises hydrolyzing a detectably-labeled probe hybridized to at least a portion of the first adapter sequence; and extending the respective one of the plurality of extension primers comprises extending an extension primer hybridized to at least a portion of the second adapter sequence.

Optionally, wherein the plurality of nucleic acid molecules further comprise a plurality of adapter molecules; and each of the plurality of adapter molecules comprises a first adapter sequence directly linked to a second adapter sequence; wherein the method further comprises contacting a plurality of hybridizing oligonucleotides with the plurality of nucleic acid molecules; wherein each of the plurality of hybridizing oligonucleotides comprises a sequence complementary to a contiguous domain of a respective one of the plurality of adapter molecules.

Optionally, each of the plurality of hybridizing oligonucleotides comprises a blocker nucleic acid domain; subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; and hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain.

Optionally, the contiguous domain comprises a portion of the first adapter sequence and a portion of the second adapter sequence directly adjacent to each other.

Optionally, the first signal is a first fluorescent signal, and the signals detected upon the extension reactions are fluorescent signals comprising the first fluorescent signal.

Optionally, each of the plurality of detectably-labeled probes further comprises a second labeled nucleic acid domain comprising a second label; the first labeled nucleic acid domain is a first reporter domain; the second labeled nucleic acid domain is a first quencher domain; and the plurality of detectably-labeled probes are hydrolyzed to release at least one of the first label or the second label.

Optionally, each of the plurality of detectably-labeled probes further comprises a second labeled nucleic acid domain comprising a second label; the first label and the second label are two spectrally similar or identical reporters; and the plurality of detectably-labeled probes are hydrolyzed to release at least one of the first label or the second label.

Optionally, the first labeled nucleic acid domain is a first reporter domain; each of the plurality of detectably-labeled probes comprises a quenching nucleotide that quenches an energy from the first label in an excited state; and the plurality of detectably-labeled probes are hydrolyzed to release at least one of the quenching nucleotide or the first label.

Optionally, each of the plurality of hybridizing oligonucleotides further comprises a third labeled nucleic acid domain comprising a third label; subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; hydrolyzation of each of the plurality of hybridizing oligonucleotides hybridized to the contiguous domain and hydrolyzation of each of the plurality of detectably-labeled probes hybridized to the first adapter sequence of each of the plurality of adapter molecules are blocked by a blocking moiety in the blocker nucleic acid domain; and the plurality of hybridizing oligonucleotides are partially hydrolyzed; wherein the method further comprises detecting a second signal produced as a result of partially hydrolyzing the plurality of hybridizing oligonucleotides; and the first signal and the second signal are distinguishably detected.

Optionally, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; the second reporter domain and the second quencher domain are linked by the blocker nucleic acid domain; and the plurality of hybridizing oligonucleotides are partially hydrolyzed to release at least one of the third label or the fourth label.

Optionally, each of the plurality of hybridizing oligonucleotides further comprises a fourth labeled nucleic acid domain comprising a fourth label; the third label and the fourth label are two spectrally similar or identical reporters; and the plurality of hybridizing oligonucleotides are partially hydrolyzed to release at least one of the third label or the fourth label.

Optionally, the third labeled nucleic acid domain is a third reporter domain; each of the plurality of hybridizing oligonucleotides comprises a quenching nucleotide that quenches an energy from the third label in an excited state; and the plurality of hybridizing oligonucleotides are partially hydrolyzed to release at least one of the quenching nucleotide or the third label.

Optionally, the first signal is a first fluorescent signal, the second signal is a second fluorescent signal, and the signals detected upon the extension reactions are fluorescent signals comprising the first fluorescent signal and the second fluorescent signal.

Optionally, each of the plurality of hybridizing oligonucleotides comprises a third labeled nucleic acid domain comprising a third label; and subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; wherein the method further comprises producing an extension product of each of the plurality of adapter molecules by extending a respective one of the plurality of extension primers in the extension reactions with the polymerase; hydrolyzing each of the plurality of hybridizing oligonucleotides hybridized to the contiguous domain and hydrolyzing each of the plurality of detectably-labeled probes hybridized to the respective one of the adapter molecules, during producing an extension product of each of the plurality of adapter molecules; detecting a second signal produced as a result of hydrolyzing the plurality of hybridizing oligonucleotides; and the first signal and the second signal are distinguishably detected.

Optionally, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; and the plurality of hybridizing oligonucleotides are hydrolyzed to release at least one of the third label or the fourth label.

Optionally, each of the plurality of hybridizing oligonucleotides further comprises a fourth labeled nucleic acid domain comprising a fourth label; the third label and the fourth label are two spectrally similar or identical reporters; and the plurality of hybridizing oligonucleotides are hydrolyzed to release at least one of the third label or the fourth label.

Optionally, the third labeled nucleic acid domain is a third reporter domain; each of the plurality of hybridizing oligonucleotides comprises a quenching nucleotide that quenches an energy from the third label in an excited state; and the plurality of hybridizing oligonucleotides are hydrolyzed to release at least one of the quenching nucleotide or the third label.

Optionally, the first signal is a first fluorescent signal, the second signal is a second fluorescent signal, and the signals detected upon the extension reactions are fluorescent signals comprising the first fluorescent signal and the second fluorescent signal.

Optionally, the first adapter sequence comprises a first flow cell binding sequence; the second adapter sequence comprises a second flow cell binding sequence; wherein, hydrolyzing each of the plurality of detectably-labeled probes comprises hydrolyzing a detectably-labeled probe hybridized to at least a portion of the first flow cell binding sequence; and extending the respective one of the plurality of extension primers comprises extending an extension primer hybridized to at least a portion of the second flow cell binding sequence.

Optionally, the first adapter sequence comprises a first sequencing primer binding sequence; the second adapter sequence further comprises a second sequencing primer binding sequence; the first sequencing primer binding sequence is directly linked to the second sequencing primer binding sequence; and the contiguous domain comprises a portion of the first sequencing primer binding sequence and a portion of the second sequencing primer binding sequence directly adjacent to each other.

Optionally, the method further comprises preparing for solid phase attachment by diluting the plurality of target nucleic acid fragments to a predetermined concentration.

Optionally, the plurality of target nucleic acid fragments are a plurality of target double-strand DNA fragments; wherein, prior to producing the extension product of each of the plurality of target nucleic acid fragments, the method further comprises denaturing the plurality of target double-strand DNA fragments.

In another aspect, the present disclosure provides a method of quantitating a plurality of nucleic acid molecules comprising a plurality of target nucleic acid fragments, the method comprising contacting a plurality of extension primers and a plurality of nucleic acid binding dye molecules with the plurality of nucleic acid molecules; producing an extension product of each of the plurality of target nucleic acid fragments by extending a respective one of the plurality of extension primers with a polymerase; subsequent to producing the extension product of each of the plurality of target nucleic acid fragments, measuring a signal produced by the plurality of nucleic acid binding dye molecules; and estimating an average size of the plurality of target nucleic acid fragments based on a number of the plurality of target nucleic acid fragments and the signal produced by the plurality of nucleic acid binding dye molecules.

Optionally, the method further comprises contacting a plurality of detectably-labeled probes and the plurality of extension primers with the plurality of nucleic acid molecules, wherein each of the plurality of detectably-labeled probes comprises a first labeled nucleic acid domain comprising a first label; producing the extension product of each of the plurality of target nucleic acid fragments by extending the respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating the number of the plurality of target nucleic acid fragments based on signals detected upon extension reactions.

Optionally, estimating an average size of the plurality of target nucleic acid fragments comprises determining a correlation factor using signals respectively produced by a plurality of reference nucleic acid libraries; estimating the average size of the plurality of target nucleic acid fragments according to the following Equation:

$$Average size = \frac{S}{correlation factor \times N} ;$$

wherein S stands for the signal produced by the plurality of nucleic acid binding dye molecules, and N stands for the number of the plurality of target nucleic acid fragments.

Optionally, the plurality of nucleic acid binding dye molecules are a plurality of double-stranded nucleic acid intercalating dye molecules.

Optionally, the plurality of nucleic acid binding dye molecules are a plurality of single-stranded nucleic acid dye molecules.

Optionally, a respective one of the plurality of nucleic acid binding dye molecules is a fluorescence dye selected from the group consisting of ethidium bromide, SYBR Green, SYBR Gold, SYBR Safe, GelRed, GelGreen, and Diamond™ Nucleic Acid Dye.

In another aspect, the present disclosure provides a method of sequencing a nucleic acid sample, comprising generating a plurality of nucleic acid molecules comprising a plurality of target nucleic acid fragments using a nucleic acid sample; contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; calculating a number of the plurality of target nucleic acid fragments based on signals detected upon a single cycle of extension reactions; diluting the plurality of target nucleic acid fragments to a predetermined concentration; and sequencing at least one portion of the plurality of target nucleic acid fragments.

In another aspect, the present disclosure provides a method for quantitating a plurality of nucleic acid molecules comprising a plurality of target nucleic acid fragments and a plurality of adapter molecules, the method comprising contacting a plurality of detectably-labeled probes, a plurality of extension primers, and a plurality of hybridizing oligonucleotides with the plurality of nucleic acid molecules, wherein each of the a plurality of detectably-labeled probes comprises a first labeled nucleic acid domain comprising a first label, and each of the a plurality of hybridizing oligonucleotides comprises a blocker nucleic acid domain; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; and detecting a first signal produced as a result of hydrolyzing the plurality of detectably-labeled probes.

Optionally, each of the plurality of hybridizing oligonucleotides comprises a sequence complementary to a contiguous domain of a respective one of the plurality of adapter molecules; subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; each of the plurality of target nucleic acid fragments comprises a first adapter sequence, a target insert sequence, and a second adapter sequence, the first adapter sequence is linked to the second adapter sequence through the target insert sequence; each of the plurality of adapter molecules comprises a first adapter sequence directly linked to a second adapter sequence; hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain.

Optionally, the contiguous domain comprises a portion of the first adapter sequence and a portion of the second adapter sequence directly adjacent to each other.

Optionally, each of the plurality of hybridizing oligonucleotides further comprises a third labeled nucleic acid domain comprising a third label; subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain; and the plurality of hybridizing oligonucleotides are partially hydrolyzed; wherein the method further comprises detecting a second signal produced as a result of partially hydrolyzing the plurality of hybridizing oligonucleotides; and the first signal and the second signal are distinguishably detected.

Optionally, the first signal is a first fluorescent signal, the second signal is a second fluorescent signal, and the signals detected upon the single cycle of extension reactions are fluorescent signals comprising the first fluorescent signal and the second fluorescent signal.

Optionally, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; the second reporter domain and the second quencher domain are linked by the blocker nucleic acid domain; and the plurality of hybridizing oligonucleotides are partially hydrolyzed to release at least one of the third label or the fourth label.

Optionally, each of the plurality of hybridizing oligonucleotides further comprises a fourth labeled nucleic acid domain comprising a fourth label; the third label and the fourth label are two spectrally similar or identical reporters; and the plurality of hybridizing oligonucleotides are partially hydrolyzed to release at least one of the third label or the fourth label.

Optionally, the third labeled nucleic acid domain is a third reporter domain; each of the plurality of hybridizing oligonucleotides comprises a quenching nucleotide that quenches an energy from the third label in an excited state; and the plurality of hybridizing oligonucleotides are partially hydrolyzed to release at least one of the quenching nucleotide or the third label.

In another aspect, the present disclosure provides a mixture, comprising a plurality of nucleic acid molecules comprising a plurality of target nucleic acid fragments; a plurality of detectably-labeled probes; and a plurality of hybridized adapter molecules; wherein each of the plurality of detectably-labeled probes comprises a first labeled nucleic acid domain comprising a first label; each of the plurality of target nucleic acid fragments comprises a first adapter sequence, a target insert sequence, and a second adapter sequence; the first adapter sequence is linked to the second adapter sequence through the target insert sequence; at least a portion of each of the plurality of detectably-labeled probes is complementary to at least a portion of the first adapter sequence; wherein each of the plurality of hybridized adapter molecules comprises the first adapter sequence; the second adapter sequence; and a partially hydrolyzed hybridizing oligonucleotide, at least a portion of which is hybridized to a domain of a respective one of a plurality of adapter molecules; wherein the first adapter sequence is directly linked to the second adapter sequence; and the domain comprises a portion of the first adapter sequence and a portion of the second adapter sequence directly adjacent to each other.

Optionally, each of the plurality of hybridized adapter molecules further comprises one of the plurality of detectably-labeled probes, at least a portion of which is hybridized to at least a portion of the first adapter sequence; and one of a plurality of extension primers, at least a portion of which is hybridized to at least a portion of the at least a portion of the second adapter sequence.

Optionally, each of the plurality of detectably-labeled probes further comprises a second labeled nucleic acid domain comprising a second label; the first labeled nucleic acid domain is a first reporter domain; and the second labeled nucleic acid domain is a first quencher domain.

Optionally, each of the plurality of detectably-labeled probes further comprises a second labeled nucleic acid domain comprising a second label; the first label and the second label are two spectrally similar or identical reporters.

Optionally, the first labeled nucleic acid domain is a first reporter domain; and each of the plurality of detectably-labeled probes comprises a quenching nucleotide that quenches an energy from the first label in an excited state.

Optionally, the first adapter sequence comprises a first flow cell binding sequence; at least a portion of each of the plurality of detectably-labeled probes is complementary to at least a portion of the first flow cell binding sequence.

Optionally, the mixture further comprises a plurality of extension primers; wherein at least a portion of each of the plurality of extension primers is complementary to at least a portion of the second adapter sequence.

Optionally, the second adapter sequence comprises a second flow cell binding sequence; and at least a portion of each of the plurality of extension primers is complementary to at least a portion of the second flow cell binding sequence.

Optionally, the mixture further comprises a plurality of hybridizing oligonucleotides; wherein each of the plurality of hybridizing oligonucleotides comprises a blocker nucleic acid domain.

Optionally, each of the plurality of hybridizing oligonucleotides further comprises a third labeled nucleic acid domain comprising a third label.

Optionally, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; and the second reporter domain and the second quencher domain are linked by the blocker nucleic acid domain.

Optionally, each of the plurality of hybridizing oligonucleotides further comprises a fourth labeled nucleic acid domain comprising a fourth label; the third label and the fourth label are two spectrally similar or identical reporters.

Optionally, the third labeled nucleic acid domain is a third reporter domain; and each of the plurality of hybridizing oligonucleotides comprises a quenching nucleotide that quenches an energy from the third label in an excited state.

In another aspect, the present disclosure provides a kit for quantitating a plurality of nucleic acid molecules comprising a plurality of target nucleic acid fragments, wherein each of the plurality of target nucleic acid fragments comprises a first adapter sequence, a target insert sequence, and a second adapter sequence; the first adapter sequence is linked to the second adapter sequence through the target insert sequence; wherein the kit comprises a plurality of detectably-labeled probes; a plurality of extension primers; and instructions directing a user to (1) producing an extension product of each of the plurality of target nucleic acid fragments by extending a respective one of the plurality of extension primers with a polymerase; and (2) calculating a number of the plurality of target nucleic acid fragments based on signals detected upon a single cycle of extension reactions; wherein each of the plurality of detectably-labeled probes comprises a first labeled nucleic acid domain comprising a first label; at least a portion of each of plurality of the detectably-labeled probes is complementary to at least a portion of the first adapter sequence; and at least a portion of each of the plurality of extension primers is complementary to at least a portion of the second adapter sequence.

Optionally, each of the plurality of detectably-labeled probes further comprises a second labeled nucleic acid domain comprising a second label; the first labeled nucleic acid domain is a first reporter domain; and the second labeled nucleic acid domain is a first quencher domain.

Optionally, each of the plurality of detectably-labeled probes further comprises a second labeled nucleic acid domain comprising a second label; the first label and the second label are two spectrally similar or identical reporters.

Optionally, the first labeled nucleic acid domain is a first reporter domain; and each of the plurality of detectably-labeled probes comprises a quenching nucleotide that quenches an energy from the first label in an excited state.

Optionally, the first adapter sequence comprises a first flow cell binding sequence; at least a portion of each of the plurality of detectably-labeled probes is complementary to at least a portion of the first flow cell binding sequence.

Optionally, the second adapter sequence comprises a second flow cell binding sequence; and at least a portion of each of the plurality of extension primers is complementary to at least a portion of the second flow cell binding sequence.

Optionally, the kit further comprises a plurality of hybridizing oligonucleotides; wherein each of the plurality of hybridizing oligonucleotides comprises a blocker nucleic acid domain.

Optionally, each of the plurality of hybridizing oligonucleotides further comprises a third labeled nucleic acid domain comprising a third label.

Optionally, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; and the second reporter domain and the second quencher domain are linked by the blocker nucleic acid domain.

Optionally, each of the plurality of hybridizing oligonucleotides further comprises a fourth labeled nucleic acid domain comprising a fourth label; the third label and the fourth label are two spectrally similar or identical reporters.

Optionally, the third labeled nucleic acid domain is a third reporter domain; and each of the plurality of hybridizing oligonucleotides comprises a quenching nucleotide that quenches an energy from the third label in an excited state.

Optionally, the kit further comprises a polymerase.

Optionally, the kit further comprises a plurality of nucleic acid binding dye molecules.

### BRIEF DESCRIPTION OF THE FIGURES

The following drawings are merely examples for illustrative purposes according to various disclosed embodiments and are not intended to limit the scope of the present invention.

FIGS. 1A to 1D illustrate several steps of a method of quantitating a plurality of nucleic acid molecules in some embodiments according to the present disclosure.

FIGS. 2A to 2D illustrate several steps of a method of quantitating a plurality of nucleic acid molecules in some embodiments according to the present disclosure.

FIGS. 3A to 3B illustrate several steps of a method of quantitating a plurality of nucleic acid molecules in some embodiments according to the present disclosure.

FIGS. 4A to 4D illustrate several steps of a method of quantitating a plurality of nucleic acid molecules in some embodiments according to the present disclosure.

FIGS. 5A to 5E illustrate several steps of a method of quantitating a plurality of nucleic acid molecules in some embodiments according to the present disclosure.

FIGS. 6A to 6D illustrate several steps of a method of quantitating a plurality of nucleic acid molecules in some embodiments according to the present disclosure.

FIGS. 7A to 7D illustrate several steps of a method of quantitating a plurality of nucleic acid molecules in some embodiments according to the present disclosure.

FIG. 8 illustrates a linear correlation between the relative fluorescent units and template concentration.

FIG. 9 illustrates that only DNA strands with the binding sites for the extension primer and the detectably-labeled probe are able to be detected.

FIGS. 10A and 10B illustrate the correlation between the NGS read number and calculated concentrations of libraries obtained using the 5400 Fragment Analyzer System or the method according to the present disclosure.

FIG. 11 illustrates the correlation between the relative fluorescent units detected using a Qubit 4 Fluorometer and the template concentration.

FIGS. 12A to 12D illustrate several steps of a method of estimating an average size of a plurality of target nucleic acid fragments in some embodiments according to the present disclosure.

FIG. 13 illustrates fluorescence values for the plurality of target nucleic acid fragments with inserts of different lengths.

FIG. 14 illustrates a correlation between fluorescence values obtained at CY3 channel and nucleic acid molarities in the libraries.

FIG. 15 illustrates a correlation between fluorescence values obtained at VIC channel and nucleic acid molarities for “control reference fragments”.

FIG. 16 illustrates the correlation between known fragment sizes and calculated fragment sizes.

### DETAILED DESCRIPTION

The disclosure will now be described more specifically with reference to the following embodiments. It is to be noted that the following descriptions of some embodiments are presented herein for purpose of illustration and description only. It is not intended to be exhaustive or to be limited to the precise form disclosed.

In next generation nucleic acid sequencing, nucleic acid fragments having adapter sequences at ends of the nucleic acid fragments are used. The nucleic acid fragments having adapter sequences at ends are amplified, and the amplified products are immobilized on a flow cell for sequencing. The adapter sequences adapt the nucleic acid fragments to the needs of the sequencer. In a nucleic acid sequencing sample, not all nucleic acid fragments contain adapters on each end. Accordingly, two distinct primers, each specific to one of the adapters, are used in an amplification step to enrich for fragments with two different adapters on their ends. The enriched collection of nucleic acid fragments with adapters on both ends is typically referred to as a library. In some circumstances, a library can have only one attached adapter. The library may be immobilized on a solid support such that a spatial distance between library elements (e.g., target nucleic acid fragments having an insert between adapters) allows for detection and recognition of each element.

Each individual library element immobilized on the surface of the solid support is amplified to increase in number to allow efficient detection of fluorophores as the fragment is being sequenced. The amplification in this step is typically referred to as bridge amplification, the result of which is typically referred to as a cluster. Other sequences methods, such as Ion Torrent and Genapsys, use beads to fix and amplify individual library molecules. During amplification, a cluster of nucleic acid fragments of the same sequence is generated on the solid support. Ideally, the cluster is homogeneous and does not contain heterogeneous fragments from any other library elements, in order to achieve a good sequencing result. If clusters are too close to each other or overlapping, image analysis software may have difficulty distinguishing the boundaries of the clusters and combine them into a single feature for data extraction. Since data from this cluster is derived from two different nucleic acid fragments with two different sequences, the software may not be able to determine the sequences accurately. Thus, the spatial distance between library elements is critical to keep clusters apart from each other. When clusters are properly spaced apart, each cluster may be analyzed separately to achieve an accurate sequencing result. However, when the clusters are too far apart, the sequencing becomes inefficient. The spatial distance between library elements is mainly determined by concentration of each individual library element, library quantitation (i.e., determination of the concentration of the library elements) is critical for achieving a more accurate and efficient sequencing process.

Accordingly, the present disclosure provides, inter alia, a method for quantitating a plurality of nucleic acid molecules comprising a plurality of target nucleic acid fragments. In some embodiments, the method includes contacting detectably-labeled probes and extension primers with the plurality of nucleic acid molecules, wherein each of the detectably-labeled probes comprises a first labeled nucleic acid domain comprising a first label; extending each of the extension primers with a polymerase to produce an extension product of a respective one of the plurality of target nucleic acid fragments; hydrolyzing each of the detectably-labeled probes hybridized to the respective one of the target nucleic acid fragments during extending each of the extension primers with the polymerase; and detecting a first signal produced as a result of hydrolyzing the detectably-labeled probes.

Detectably-Labeled Probes

Various appropriate formats of detectably-labeled probes may be used in the present disclosure. As used herein, the term “probe” refers to an oligonucleotide (or other nucleic acid sequence) which can form a duplex structure with a region of a target nucleic acid (or amplicon derived from such target nucleic acid), due to partial or complete complementarity of at least one sequence in the probe with a sequence in the target nucleic acid under suitable conditions. As defined herein, the probe can further comprise non-nucleotide fragments or components, e.g. a non-nucleotide linker. As discussed herein, the probe can be labeled or unlabeled. The 3′-terminus of the probe optionally can be designed to prohibit extension (non-extendible). This can be achieved by using non-complementary bases or by adding a chemical moiety such as biotin or a phosphate group to the 3′-hydroxyl group of the last nucleotide, which can, depending upon the selected moiety, serve a dual purpose by also acting as a label for subsequent detection or capture of the nucleic acid attached to the label. Prohibiting extension can also be achieved by removing the 3′-OH or by using a nucleotide that lacks a 3′-OH such as a dideoxynucleotide, or by adding a bulky group that blocks extension by steric hindrance.

As used herein, the term “detectably-labeled probe” means that a probe has been conjugated with a label or that a probe has some inherent characteristic (e.g., size, shape or color) that allows it to be detected without having to be conjugated to a separate label.

As user herein, the term “label” refers to any atom or molecule that can be used to provide a detectable (preferably quantifiable) effect, and that can be attached to a nucleic acid or protein. Labels include but are not limited to dyes; radiolabels such as 32P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. A label may be composed of a specific molecular weight which can be detected when removed from the probe. Labels can include or consist of nucleic acid or protein sequence, so long as the sequence comprising the label is detectable.

As user herein, the term “signal” refers to any detectable effect, such as would be caused or provided by a label or an assay reaction, e.g., an enzymatic reaction.

As used herein, the term “detectably labeled,” or “detectable label” refers to a chemical species that can be detected or can lead to a detectable response. Detectable labels in accordance with the disclosure can be linked to probe either directly or indirectly. In some embodiments, detectable labels are members of an interactive label pair. In some other embodiments, one member of the label pair is a fluorophore, and the other member of the label pair is a quencher.

In some embodiments, each of the detectably-labeled probes includes a first labeled nucleic acid domain comprising a first label and a second labeled nucleic acid domain comprising a second label. In a first exemplary format, the first labeled nucleic acid domain is a reporter domain, the second labeled nucleic acid domain is a quencher domain. Non-limiting examples of such formats include a TaqMan probe format (see, e.g., Ririe, K. M., Rasmussen, R. P., Wittwer, C. T., Anal Biochem 245: 154-160, 1997; Holland, P. M., Abramson, R. D., Watson, R., et al., Proc Natl Acad Sci USA 88: 7276-7280, 1991). In a second exemplary format, the first label and the second label are two spectrally similar or identical reporters. Non-limiting examples of such formats include an AllGlo probe format (see, e.g., US Patent Publication No. 20100240103).

In some embodiments, each of the detectably-labeled probes includes a first labeled nucleic acid domain comprising a first label. In a third exemplary format, the first labeled nucleic acid domain is a reporter domain, each of the detectably-labeled probes comprises a quenching nucleotide that quenches an energy from the first label in an excited state. Non-limiting examples of such formats include those described in Nazarenko I, Pires R, et al., Nuc Acids Res, 30: 2089-2195, 2002; Kelley S O and Barton J K., Science, 283: 375-381, 1999; and Seidel C A M, Schulz A, and Sauer M H M. J Phys Chem, 100: 5541-5553, 1996.

Polymerase

As used herein, the term “polymerase” and its variants comprise any enzyme that can catalyze the polymerization of nucleotides (including analogs thereof) into a nucleic acid strand. Typically but not necessarily such nucleotide polymerization can occur in a template-dependent fashion. The template strand and synthesized nucleic acid strand can independently be either DNA or RNA. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, homologs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases (such as for example Thermus aquaticus (Taq) DNA polymerase I, and E. coli DNA polymerase). The term “polymerase” and its variants, as used herein, also refers to fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. The polymerase is a polypeptide or protein containing sufficient amino acids to carry out a desired enzymatic function of the polymerase. The polymerase need not contain all of the amino acids found in the native enzyme but only those which are sufficient to allow the polymerase to carry out a desired catalytic activity. Catalytic activities include, for example, 5′-3′ polymerization, and 5′-3′ exonuclease activities.

In some embodiments, the polymerase used in extending each of the extension primers to produce an extension product of the respective one of the plurality of target nucleic acid fragments is a polymerase having polymerase activity and 5′-3′ exonuclease activity. Optionally, the polymerase used in extending each of the extension primers to produce an extension product of the respective one of the plurality of target nucleic acid fragments is a polymerase having polymerase activity and 5′-3′ exonuclease activity, but does not have 3′-5′ exonuclease activity. Examples of polymerases having polymerase activity and 5′-3′ exonuclease activity include Taq polymerase (such as Taq DNA polymerase I) and Tth polymerase (such as Tth DNA polymerase). By means of this 5′→3′ exonuclease activity the DNA polymerase may nucleolytically attack the labeled 5′-termini of the detectably-labeled probes that are hybridized to the respective one of the target nucleic acid fragments, resulting in a progressive degradation of such detectably-labeled probes. As a result, the first label L1 (e.g., a reporter moiety) and the second label L2 (e.g., a quencher moiety) are spaced apart from each other, e.g., by a distance greater than the minimum quenching distance, thereby allowing the reporter moiety to provide a fluorescence signal.

As used herein, the term “extension condition” refer to a condition under which a primer that hybridizes to a template nucleic acid is extended by a polymerase during an extension step. Those of skill in the art will appreciate that such conditions can vary, and are generally influenced by ionic strength and temperature.

Accordingly, in some embodiments, the present disclosure provides a method for quantitating a plurality of nucleic acid molecules comprising a plurality of target nucleic acid fragments. In some embodiments, the method includes contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating a number of the plurality of target nucleic acid fragments based on signals detected upon a single cycle of extension reactions. Optionally, the first signal is a first fluorescent signal, and the signals detected upon the single cycle of extension reactions are fluorescent signals detected upon the single cycle of extension reactions. In the following Embodiments, fluorescent signals are used as non-limiting examples for illustrating the method, however, the present method is not limited to the use of fluorescent signals.

Primer

As used herein, the “primer” is a nucleic acid that can hybridize to a target or template nucleic acid and permit extension or elongation using, e.g., a nucleotide incorporating biocatalyst, such as a polymerase under appropriate reaction conditions. Such conditions typically include the presence of one or more deoxyribonucleoside triphosphates and the nucleotide incorporating biocatalyst, in a suitable buffer (“buffer” includes substituents which are cofactors, or which affect pH, ionic strength, etc.), and at a suitable temperature. A primer nucleic acid is typically a natural or synthetic oligonucleotide (e.g., a single-stranded oligodeoxyribonucleotide, etc.). Although other primer nucleic acid lengths are optionally utilized, they typically comprise hybridizing regions that range from about 6 to about 100 nucleotides in length. Short primer nucleic acids generally require lower temperatures to form sufficiently stable hybrid complexes with template nucleic acids. A primer nucleic acid that is at least partially complementary to a subsequence of a template nucleic acid is typically sufficient to hybridize with the template for extension to occur. The design of suitable primers for, e.g., the amplification of a given target sequence is well known in the art and described in the literature cited herein. A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or otherwise known in the art. Optionally, the primer (e.g., an extension primer) binds to the target or template nucleic acid specifically. Optionally, the primer (e.g., an extension primer) binds to the target or template nucleic acid non-specifically, however, under the extension condition, still permitting extension or elongation. In one example, a specifically designed extension primer that binds to the target or template nucleic acid specifically is used in the method according to the present disclosure. In another example, one or more of the plurality of target nucleic acid fragments may be used as extension primers in the method according to the present disclosure.

Blocking Moiety

As used herein, the term “blocking moiety” refers to a moiety that typically prevents the extension of a nucleic acid (e.g., an extension primer). For example, the blocking moiety may be a moiety that prevents extension of an extension primer by a polymerase having a polymerase activity and a 5′-3′ exonuclease activity. Optionally, the blocking moiety is a naturally-occurring nucleotide. Optionally, the blocking moiety is a modified nucleotide. Examples of blocking moieties include a peptide nucleic acid, a locked nucleic acid, a nucleotide linked to a minor groove binder, d(2-am)ATP, 5-methylcytosine, a phosphorothioate nucleotide, a hydrolysis-resistant and extension-blocking nucleotide, and a nucleotide linked to polyethylene glycol.

Nucleic Acid Binding Dye Molecules

Various appropriate nucleic acid binding dye molecules may be used in the present methods, kits, and mixtures. Optionally, the plurality of nucleic acid binding dye molecules are a plurality of double-stranded nucleic acid intercalating dye molecules. Optionally, the plurality of nucleic acid binding dye molecules are a plurality of single-stranded nucleic acid dye molecules. Examples of nucleic acid binding dye molecules include ethidium bromide, SYBR Green (e.g., SYBR Green I, SYBR Green II), SYBR Gold, SYBR Safe, SYBR GreenEr, Gel Red, Gel Green, and Diamond™ Nucleic Acid Dye, actinomycin D, psoralen, 4′-aminomethyl-4,5′,8-trimethylpsoralen (AMT), Hoechst 33258, EvaGreen dye, OliGreen, RiboGreen, LC Green, LC Green Plus, BOXTO, BEBO, SYBR DX, SYTO9, SYTOX Blue, SYTOX Green, SYTOX Orange, SYTO dyes (e.g., SYTO82 and SYTO59), POPO-1, POPO-3, BOBO-1, BOBO-3, YOYO-1, YOYO-3, TOTO-1, TOTO-3, PO-PRO-1, BO-PRO-1, YO-PRO-1, TO-PRO-1, JO-PRO-1, PO-PRO-3, LO-PRO-1, BO-PRO-3, YO-PRO-3, TO-PRO-3, TO-PRO-5, Ethidium Homodimer-1, Ethidium Homodimer-2, Ethidium Homodimer-3, propidium iodide, various Hoechst dyes, DAPI, ResoLight, Chromofy, acridine homodimer, and propidium iodide.

### Embodiment I

In some embodiments, each of the detectably-labeled probes includes a first labeled nucleic acid domain comprising a first label and a second labeled nucleic acid domain comprising a second label. Optionally, the first labeled nucleic acid domain is a first reporter domain; the second labeled nucleic acid domain is a first quencher domain; and the detectably-labeled probes are hydrolyzed to release at least one of the first label or the second label.

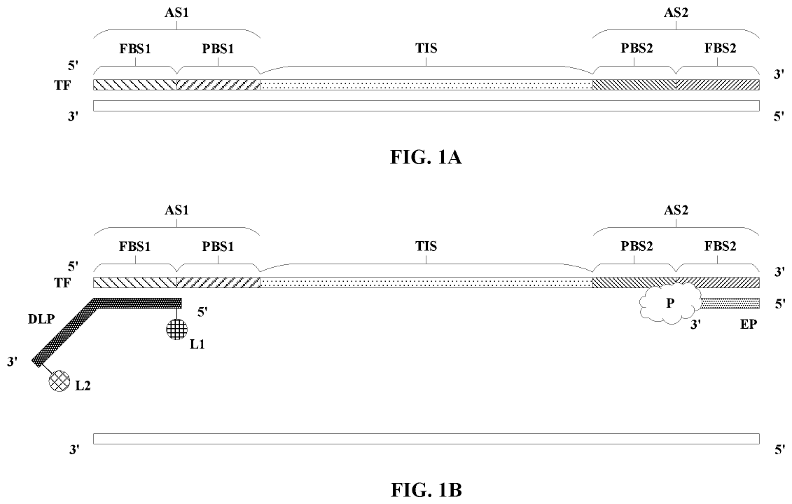

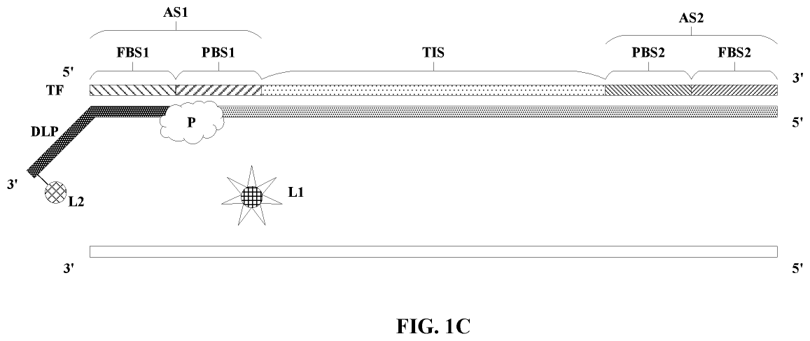

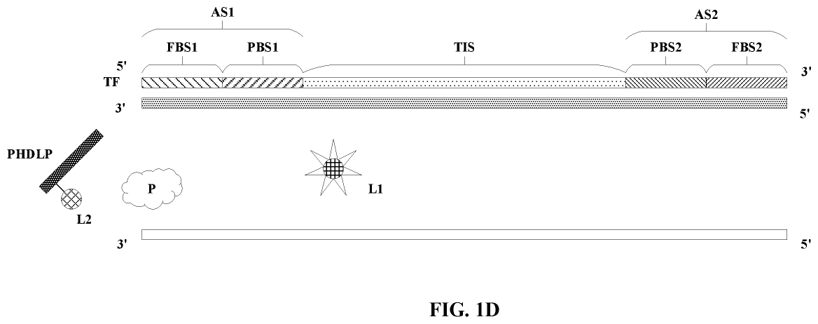

FIGS. 1A to 1D illustrate several steps of a method of quantitating a plurality of nucleic acid molecules in some embodiments according to the present disclosure. Referring to FIG. 1A, a target nucleic acid fragment TF is shown. In FIG. 1A, a DNA molecule is shown as a non-limiting example for illustrating the present method. The present method is suitable for quantitating various types of nucleic acid molecules including single-strand nucleic acid molecules (e.g., RNA molecules) and double-strand nucleic acid molecules (e.g., DNA molecules).

In some embodiments, the target nucleic acid fragment TF includes a first adapter sequence AS1, a target insert sequence TIS, and a second adapter sequence AS2. In one example, the first adapter sequence AS1 is linked to the second adapter sequence AS2 through the target insert sequence TIS. The adapter sequences may be ones that are used in various NGS sequencing technologies. In one example, the first adapter sequence AS1 includes a first sequencing primer binding sequence PBS1 at its 3′ end; and a first flow cell binding sequence FBS1 at its 5′ end. In another example, the second adapter sequence AS2 includes a second sequencing primer binding sequence PBS2 at its 5′ end; and a second flow cell binding sequence FBS2 at its 3′ end. Optionally, the adapter sequences further include other sequences, for example, index primer binding sequences. In one example, the first adapter sequence AS1 further includes a first index primer binding sequence between the first flow cell binding sequence FBS1 and the first sequencing primer binding sequence PBS1. In another example, the second adapter sequence AS2 further includes a second index primer binding sequence between the second sequencing primer binding sequence PBS2 and the second flow cell binding sequence FBS2.

As used herein, the term “adapter sequence” refers to nucleotide sequences added to one or both ends of a sample nucleic acid fragment in order for the sample nucleic acid fragment to be sequenced. Sequencing can occur either directly (without amplification) or after amplification using primers wherein either the sequencing adapter or primers comprise one of a primer binding site, a capture oligonucleotide binding site, a polymerase binding site, a sequencing bar-code, an indexing sequence, a random nucleotide, a unique molecular identifier (UMI), a sequencing flow-cell binding site, or any combinations thereof.

Examples of flow cell binding sequences include:

(SEQ ID NO: 1)
5′-AATGATACGGCGACCACCGA-3′
(SEQ ID NO: 2)
5′-AATGATACGGCGACCACCGAGATCTACAC-3′
(SEQ ID NO: 3)
5′-TCGTATGCCGTCTTCTGCTTG-3′
(SEQ ID NO: 4)
5′-ATCTCGTATGCCGTCTTCTGCTTG-3′

Examples of sequencing primer binding sequences include:

(SEQ ID NO: 5)
5′-ACACTCTTTCCCTACACGACGCTCTTCCGATCT-3′
(SEQ ID NO: 6)
5′-GTGACTGGAGTTCAGACGTGTGCTCTTCCGATCT-3′

Examples of index primer binding sequences include:

(SEQ ID NO: 7)
5′-GATCGGAAGAGCACACGTCTGAACTCCAGTCAC-3′

In some embodiments, the method includes a step of contacting detectably-labeled probes and extension primers with the plurality of nucleic acid molecules, wherein each of the detectably-labeled probes includes a first reporter nucleic acid domain and a first quencher nucleic acid domain. Referring to FIG. 1B, detectably-labeled probes DLP are added to a solution containing the plurality of nucleic acid molecules. The plurality of nucleic acid molecules include the plurality of target nucleic acid fragments TF.

In some embodiments, the plurality of target nucleic acid fragments are strands of a plurality of target double-strand DNA fragments. Optionally, the method further includes denaturing the plurality of target double-strand DNA fragments and annealing the extension primers and detectably-labeled probes to the denatured DNA fragments (e.g., prior to extending each of the extension primers with a polymerase as discussed in a section below).

In some embodiments, the extension primers includes at least one of the plurality of nucleic acid molecules that non-specifically binds to a respective one of the plurality of target nucleic acid fragments. Optionally, all extension primers are multiple ones of the plurality of nucleic acid molecules in the library, each of which non-specifically binds to the respective one of the plurality of target nucleic acid fragments, e.g., no exogenous extension primers were added into the reaction. Optionally, the extension primers includes at least one of the plurality of target nucleic acid fragments that non-specifically binds to the respective one of the plurality of target nucleic acid fragments. Optionally, all extension primers are multiple ones of the plurality of target nucleic acid fragments that non-specifically binds to the respective one of the plurality of target nucleic acid fragments.

In some embodiments, as illustrated in FIG. 1B, the target nucleic acid fragment TF is one strand of a double-strand nucleic acid molecule, and the double-strand nucleic acid molecule is denatured (e.g., by heating) to separate the target nucleic acid fragment TF from its complementary strand.

In some embodiments, the plurality of target nucleic acid fragments TF are a plurality of target single-strand nucleic acid fragments such as RNA fragments. Optionally, denaturation of the target nucleic acid fragments is not required when the plurality of target nucleic acid fragments TF are a plurality of target single-strand nucleic acid fragments. The extension primers and detectably-labeled probes are annealed to the plurality of target single-strand nucleic acid fragments without a denaturing step.

The detectably labeled probe in some embodiments includes a first labeled nucleic acid domain and a second labeled nucleic acid domain. Referring to FIG. 1B, the detectably labeled probe DLP in some embodiments includes a first labeled nucleic acid domain having a first label L1, and a second labeled nucleic acid domain having a second label L2. The labels may be conjugated to the detectably labeled probe. Optionally, the first labeled nucleic acid domain is directly linked to the second labeled nucleic acid domain.

In some embodiments, the first labeled nucleic acid domain and the second labeled nucleic acid domain are two different domains selected from a first reporter nucleic acid domain and a first quencher nucleic acid domain, i.e., the detectably labeled probe in some embodiments includes a first reporter nucleic acid domain and a first quencher nucleic acid domain. Optionally, the first labeled nucleic acid domain is a first reporter nucleic acid domain; and the second labeled nucleic acid domain is a first quencher nucleic acid domain. Optionally, the first labeled nucleic acid domain is a first quencher nucleic acid domain; and the second labeled nucleic acid domain is a first reporter nucleic acid domain. Optionally, the first label L1 is a reporter moiety, and the second label L2 is a quencher moiety. Optionally, the first label L1 is a quencher moiety, and the second label L2 is a reporter moiety.

As used herein, the term “reporter moiety” refers to a moiety capable of generating a fluorescence signal (e.g., a fluorophore). A “quencher moiety” refers to a moiety capable of absorbing the fluorescence energy of an excited reporter molecule, thereby quenching the fluorescence signal that would otherwise be released from the excited reporter moiety. In order for a quencher moiety to quench an excited fluorophore, it is often advantageous that the quencher moiety is within a minimum quenching distance of the excited reporter moiety at some time starting from the excitation of the reporter moiety, but prior to the reporter moiety releasing the stored fluorescence energy. In proximity based quenching applications, the reporter and quencher moieties are positioned sufficiently close to each other such that whenever the reporter moiety is excited, the energy of the excited state transfers to the quencher moiety where it either dissipates nonradiatively or is emitted at a different emission frequency than that of the reporter moiety. Several non-radiative energy transfer mechanisms work over shorter distances and are appropriate for proximity based quenching applications.

In some embodiments, at least a portion of each of the detectably-labeled probes DLP is complementary to at least a portion of the first adapter sequence AS1. In one example, at least a portion of each of the detectably-labeled probes DLP is complementary to at least a portion of the first flow cell binding sequence FBS1. In another example, at least a portion of each of the detectably-labeled probes DLP is complementary to at least a portion of the first sequencing primer binding sequence PBS1. In another example, at least a portion of each of the detectably-labeled probes DLP is complementary to at least a portion of the first flow cell binding sequence FBS1 and at least a portion of the first sequencing primer binding sequence PBS1. Referring to FIG. 1B, in one example, a portion of each of the detectably-labeled probes DLP is complementary to at least a portion of the first flow cell binding sequence FBS1. Subsequent to or during contacting detectably-labeled probes and extension primers with the plurality of nucleic acid molecules, the detectably-labeled probe DLP is hybridized to the first adapter sequence AS1, e.g., hybridized to at least a portion of the first flow cell binding sequence FBS1 as shown in FIG. 1B. In certain embodiments, subsequent to or during contacting detectably-labeled probes and extension primers with the plurality of nucleic acid molecules, the detectably-labeled probe DLP is hybridized to at least a portion of the first sequencing primer binding sequence PBS1. In certain embodiments, subsequent to or during contacting detectably-labeled probes and extension primers with the plurality of nucleic acid molecules, the detectably-labeled probe DLP is hybridized to at least a portion of the first flow cell binding sequence FBS1 and at least a portion of the first sequencing primer binding sequence PBS1.

In some embodiments, at least a portion of each of the detectably-labeled probes DLP is non-complementary to the first adapter sequence AS1. As shown in FIG. 1B, in one example, at least the second labeled nucleic acid domain having the second label L2 is non-complementary to the first adapter sequence AS1, and at least the first labeled nucleic acid domain having the second label L1 is complementary to first adapter sequence AS1.

In some embodiments, at least a portion of each of the extension primers EP is complementary to at least a portion of the second adapter sequence AS2. In one example, at least a portion of each of the extension primers EP is complementary to at least a portion of the second flow cell binding sequence FBS2. In another example, at least a portion of each of the extension primers EP is complementary to at least a portion of the second sequencing primer binding sequence PBS2. In another example, at least a portion of each of the extension primers EP is complementary to at least a portion of the second flow cell binding sequence FBS2 and at least a portion of the second sequencing primer binding sequence PBS2. Referring to FIG. 1B, in one example, a portion of each of the extension primers EP is complementary to at least a portion of the second flow cell binding sequence FBS2. Subsequent to or during contacting detectably-labeled probes and extension primers with the plurality of nucleic acid molecules, the extension primer EP is hybridized to the second adapter sequence AS2, e.g., hybridized to at least a portion of the second flow cell binding sequence FBS2 as shown in FIG. 1B. In certain embodiments, subsequent to or during contacting detectably-labeled probes and extension primers with the plurality of nucleic acid molecules, the extension primers EP is hybridized to at least a portion of the second sequencing primer binding sequence PBS2.

A nucleic acid is “complementary” in relation to another nucleic acid when at least a nucleic acid segment (i.e., at least two contiguous bases) can combine in an antiparallel association or hybridize with at least a subsequence of other nucleic acid to form a duplex. The antiparallel association can be intramolecular, e.g., in the form of a hairpin loop within a nucleic acid, or intermolecular, such as when two or more single-stranded nucleic acids hybridize with one another. In the context of the present invention, for an oligonucleotide that is “fully complementary” to particular sequence, each base of the oligonucleotide is complementary to the corresponding bases in the particular sequence in an anti-parallel manner. Certain bases not commonly found in natural nucleic acids may be included in the nucleic acids of the present invention and include, for example, inosine, 7-deazaguanine locked nucleic acids, and those discussed above. In some embodiments, complementarity is not perfect (i.e., nucleic acids can be “partially complementary” rather than “fully complementary”). Stable duplexes, for example, may contain mismatched base pairs or unmatched bases.

In some embodiments, the method further includes extending each of the extension primers with a polymerase to produce an extension product of a respective one of the plurality of target nucleic acid fragments; and hydrolyzing each of the detectably-labeled probes hybridized to the respective one of the target nucleic acid fragments during extending each of the extension primers with the polymerase. Referring to FIG. 1C, in some examples, an extension primer EP, in the presence of a polymerase P and under an extension condition, is extended to produce an extension product of the respective one of the plurality of target nucleic acid fragments TF. When the extension primer EP is extended to the first labeled nucleic acid domain having the first label L1, the polymerase P attacks the 5′-termini of the first labeled nucleic acid domain which is hybridized to the respective one of the target nucleic acid fragments TF, progressively degrading the first labeled nucleic acid domain. As a result, the first label L1 is released from the detectably-labeled probe DLP. In one example, the first label L1 and the second label L2 constitute a reporter-quencher pair. In an intact detectably-labeled probe DLP, the first label L1 and the second label L2 are within a minimum quenching distance, and the energy released from an excited reported is quenched by the quencher. When the first label L1 is released from the detectably-labeled probe DLP, the first label L1 and the second label L2 are spaced apart from each other, e.g., by a distance greater than the minimum quenching distance, thereby allowing the reporter moiety (e.g., the first label L1) to provide a fluorescence signal. Optionally, the method further includes detecting a first signal produced as a result of hydrolyzing the detectably-labeled probes. Optionally, the first signal is a first fluorescent signal, and the signals detected upon the single cycle of extension reactions are fluorescent signals comprising the first fluorescent signal.

Referring to FIG. 1C, in one example, hydrolyzing each of the detectably-labeled probes DLP includes hydrolyzing the detectably-labeled probe DLP hybridized to at least a portion of the first adapter sequence FBS1; and extending each of the extension primers EP includes extending each of the extension primers EP hybridized to at least a portion of the second adapter sequence FBS2. In certain embodiments, hydrolyzing each of the detectably-labeled probes DLP includes hydrolyzing the detectably-labeled probe DLP hybridized to at least a portion of the first sequencing primer binding sequence PBS1. In certain embodiments, hydrolyzing each of the detectably-labeled probes DLP includes hydrolyzing the detectably-labeled probe DLP hybridized to at least a portion of the first adapter sequence FBS1 and at least a portion of the first sequencing primer binding sequence PBS1.

Referring to FIG. 1D, in one example, the extension primer EP continues extending to hydrolyze the hybridized portion of the detectably-labeled probe DLP hybridized to the respective one of the target nucleic acid fragments TF (e.g., replacing the entire hybridized portion of the detectably-labeled probe DLP hybridized to the respective one of the target nucleic acid fragments TF), releasing the non-hybridized portion of the detectably-labeled probe, resulting in a partially hydrolyzed detectably-labeled probe DLP. Subsequent to replacing the entire hybridized portion of the detectably-labeled probe, neither the first label L1 nor the second label L2 is attached to a nucleic acid hybridized to the respective one of the plurality of target nucleic acid fragments TF. The first label L1 and the second label L2 are spaced apart from each other, e.g., by a distance greater than the minimum quenching distance, thereby allowing the reporter moiety (e.g., the first label L1) to provide a fluorescence signal.

The term “hybridized” as applied to a polynucleotide refers to a polynucleotide in a complex that is stabilized via hydrogen bonding between bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. The hybridization reaction may constitute a step in a more extensive process, such as the initiation of a PCR reaction, or the enzymatic cleavage of a polynucleotide by a ribozyme. A sequence hybridized with a given nucleic acid is referred to as the “complement” or “reverse-complement” of the given molecule if its sequence of bases over a given region is capable of complementarily binding those of its binding partner, such that, for example, A-T, A-U, and G-C and G-U base pairs are formed.

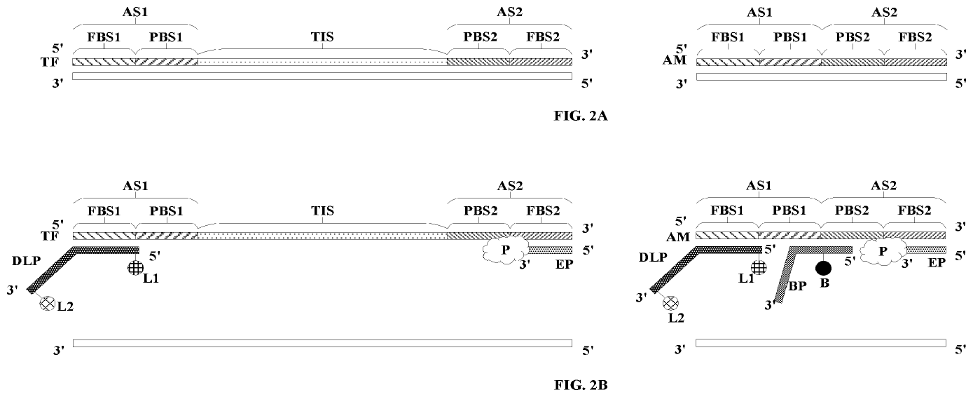

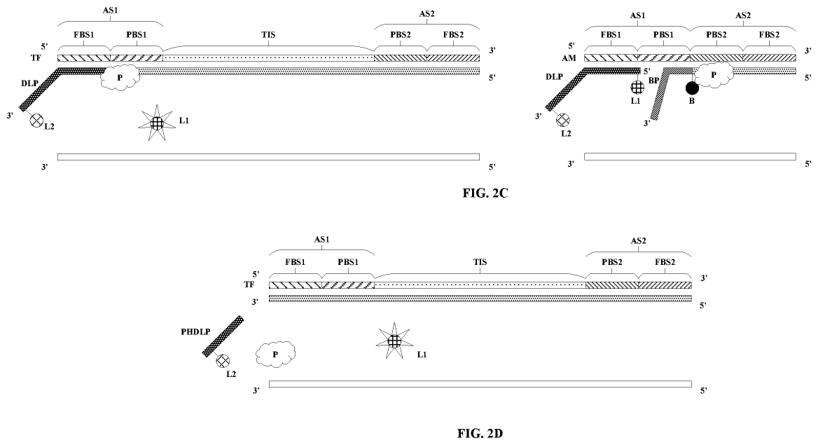

In some embodiments, the plurality of nucleic acid molecules further comprise a plurality of adapter molecules. FIGS. 2A to 2D illustrate several steps of a method of quantitating a plurality of nucleic acid molecules in some embodiments according to the present disclosure. Referring to FIG. 2A, in some embodiments, each of the adapter molecules AM includes the first adapter sequence AS1 and the second adapter sequence AS2. In one example, the first adapter sequence AS1 is directly linked to the second adapter sequence AS2 in each of the adapter molecules AM. As discussed in a previous section, the adapter sequences may be ones that are used in various NGS sequencing technologies. In one example, the first adapter sequence AS1 includes the first sequencing primer binding sequence PBS1 at its 3′ end; and the first flow cell binding sequence FBS1 at its 5′ end. In another example, the second adapter sequence AS2 includes a second sequencing primer binding sequence PBS2 at its 5′ end; and a second flow cell binding sequence FBS2 at its 3′ end. Optionally, the first sequencing primer binding sequence PBS1 is directly linked to the second sequencing primer binding sequence PBS2. Optionally, the adapter sequences further include other sequences, for example, index primer binding sequences. In one example, the first adapter sequence AS1 further includes a first index primer binding sequence between the first flow cell binding sequence FBS1 and the first sequencing primer binding sequence PBS1. In another example, the second adapter sequence AS2 further includes a second index primer binding sequence between the second sequencing primer binding sequence PBS2 and the second flow cell binding sequence FBS2.

In some embodiments, the method further includes contacting hybridizing oligonucleotides with the plurality of nucleic acid molecules. Optionally, the step of contacting hybridizing oligonucleotides with the plurality of nucleic acid molecules and the step of contacting detectably-labeled probes and extension primers with the plurality of nucleic acid molecules are performed simultaneously. In one example, the method includes providing a reagent mixture including detectably-labeled probes, extension primers, and hybridizing oligonucleotides; and contacting the reagent mixture with the plurality of nucleic acid molecules.

In some embodiments, each of the hybridizing oligonucleotides includes a sequence complementary to a contiguous domain of a respective one of the plurality of adapter molecules; and each of the hybridizing oligonucleotides includes a blocker nucleic acid domain. Referring to FIG. 2B, in some embodiments, subsequent to or during contacting hybridizing oligonucleotides BP with the plurality of nucleic acid molecules, each of the hybridizing oligonucleotides BP is hybridized to a contiguous domain of a respective one of the plurality of adapter molecules AM. Each of the hybridizing oligonucleotides BP includes a sequence complementary to the contiguous domain of a respective one of the plurality of adapter molecules AM. In one example, the contiguous domain includes a portion of the first adapter sequence AS1 and a portion of the second adapter sequence AS2 directly adjacent to each other. For example, in the contiguous domain, the portion of the first adapter sequence AS1 is directly linked to the portion of the second adapter sequence AS2. In one example, the contiguous domain includes at least a portion of the first sequencing primer binding sequence PBS1 and at least a portion the second sequencing primer binding sequence PBS2, wherein the portion of the first sequencing primer binding sequence PBS1 is directly linked to the portion the second sequencing primer binding sequence PBS2.

In some embodiments, subsequent to or during contacting the reagent mixture including detectably-labeled probes, extension primers, and hybridizing oligonucleotides with the plurality of nucleic acid molecules, at least one extension primer is hybridized to the plurality of adapter molecules AM. Optionally, subsequent to or during contacting the reagent mixture with the plurality of nucleic acid molecules, one molecule of the detectably-labeled probes DLP is hybridized to the first adapter sequence AS1 of a respective one of the plurality of target nucleic acid fragments TF and another molecule of the detectably-labeled probes DLP is hybridized to the first adapter sequence AS1 of a respective one of the plurality of adapter molecules AM; and one molecule of the extension primers EP is hybridized to the respective one of the plurality of target nucleic acid fragments TF and another molecule of the extension primers EP is hybridized to the respective one of the plurality of adapter molecules AM.

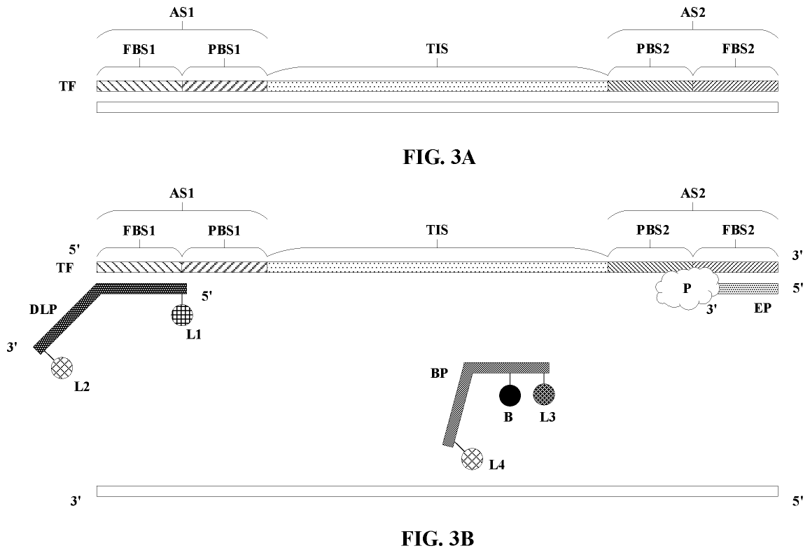

In some embodiments, subsequent to or during contacting the reagent mixture with the plurality of nucleic acid molecules, the hybridizing oligonucleotides BP are hybridized to the plurality of adapter molecules AM, e.g., each of the hybridizing oligonucleotides BP is hybridized to the contiguous domain of a respective one of the plurality of adapter molecules AM. However, the hybridizing oligonucleotides BP are not hybridized to the plurality of target nucleic acid fragments TF. FIGS. 3A to 3B illustrate several steps of a method of quantitating a plurality of nucleic acid molecules in some embodiments according to the present disclosure. Referring to FIGS. 3A to 3B, and FIGS. 2A to 2B, subsequent to or during contacting the reagent mixture including detectably-labeled probes, extension primers, and hybridizing oligonucleotides with the plurality of nucleic acid molecules, the hybridizing oligonucleotides BP only hybridizes to the adapter molecules AM, but fails to hybridize to the target nucleic acid fragments TF.

In some embodiments, the plurality of target nucleic acid fragments are a plurality of target double-strand DNA fragments; and the plurality of adapter molecules are a plurality of double-strand adapter molecules. Optionally, the method further includes, e.g., prior to extending each of the extension primers with a polymerase, denaturing the plurality of target double-strand DNA fragments and denaturing the plurality of double-strand adapter molecules, e.g., in a heating step; and annealing the extension primers and detectably-labeled probes to the denatured target DNA fragments and annealing the extension primers and the hybridizing oligonucleotides to the denatured adapter molecules. In one example, subsequent to denaturing the plurality of target double-strand DNA fragments and denaturing the plurality of double-strand adapter molecules, the hybridizing oligonucleotides only hybridizes to the adapter molecules, but fails to hybridize to the target nucleic acid fragments.

In some embodiments, the plurality of target nucleic acid fragments TF are a plurality of target single-strand nucleic acid fragments such as RNA fragments. Optionally, denaturation of the target nucleic acid fragments is not required when the plurality of target nucleic acid fragments TF are a plurality of target single-strand nucleic acid fragments.

In some embodiments, each of the hybridizing oligonucleotides includes a blocker nucleic acid domain. In one example, the blocker nucleic acid domain includes a blocking moiety.

In some embodiments, during the step of extending each of the extension primers with the polymerase, hydrolyzation of each of the hybridizing oligonucleotides BP hybridized to the contiguous domain and hydrolyzation of each of the detectably-labeled probes DLP hybridized to the first adapter sequence AS1 of each of the plurality of adapter molecules AM are blocked by the blocking moiety in the blocker nucleic acid domain. Referring to FIGS. 2B to 2D, in some examples, an extension primer EP, in the presence of a polymerase P and under an extension condition, is extended to produce an extension product of the respective one of the plurality of adapter molecules AM. When the extension primer EP is extended to a hybridizing oligonucleotide BP, the polymerase P attacks the 5′-termini of the hybridizing oligonucleotide BP which is hybridized to the respective one of the adapter molecules AM, progressively degrading the hybridizing oligonucleotide BP. When the progressive degradation continues to a blocking moiety B, the extension is blocked by the blocking moiety B.

In a sequencing library, the plurality of nucleic acid molecules include not only a plurality of target nucleic acid fragments having a target insert sequence, but often also include a plurality of adapter molecules that having no target inserts. Thus, an accurate library quantitation requires calculating a number of the plurality of target nucleic acid fragments without erroneously including the number of the plurality of adapter molecules in the calculation. The present method enables accurately calculating the number of the plurality of target nucleic acid fragments in the sequencing library because the detectably-labeled probes hybridized to the plurality of adapter molecules are not hydrolyzed during the extension step. Thus, detecting the first signal (e.g., a first fluorescent signal) produced as a result of hydrolyzing the detectably-labeled probes accurately reflects the number of the plurality of target nucleic acid fragments in the library, without the bias introduced by the plurality of adapter molecules in the library.

In some embodiments, the method further includes calculating a number of the plurality of target nucleic acid fragments based on fluorescent signals detected. Because the hydrolyzation of each of the detectably-labeled probes hybridized to the first adapter sequence of a respective one of the plurality of adapter molecules is blocked by a blocking moiety in the blocker nucleic acid domain, the step of calculating the number of the plurality of target nucleic acid fragments is based on the first signal (e.g., a first fluorescent signal).

In some embodiments, a total number of hydrolyzed detectably-labeled probes is substantially same as a total number of the plurality of target nucleic acid fragments prior to contacting the plurality of detectably-labeled probes and the plurality of extension primers with the plurality of nucleic acid molecules. As used herein, the term “substantially same” means that a difference between two values not exceeding 30% of a base value (e.g., one of the two values), e.g., not exceeding 25%, not exceeding 20%, not exceeding 15%, not exceeding 10%, not exceeding 8%, not exceeding 6%, not exceeding 4%, not exceeding 2%, not exceeding 1%, not exceeding 0.5%, not exceeding 0.1%, not exceeding 0.05%, and not exceeding 0.01%, of the base value.

In some embodiments, the total number of hydrolyzed detectably-labeled probes is correlated to an amplitude of the first signal; and the total number of the plurality of target nucleic acid fragments prior to contacting the plurality of detectably-labeled probes and the plurality of extension primers with the plurality of nucleic acid molecules is correlated to the amplitude of the first signal. Optionally, the first signal is a first fluorescent signal, and the signals detected upon the single cycle of extension reactions are fluorescent signals comprising the first fluorescent signal.

### Embodiment II

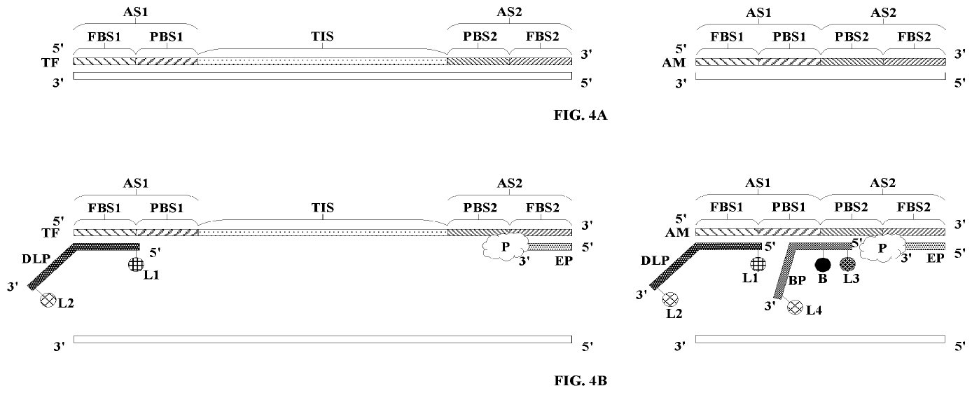

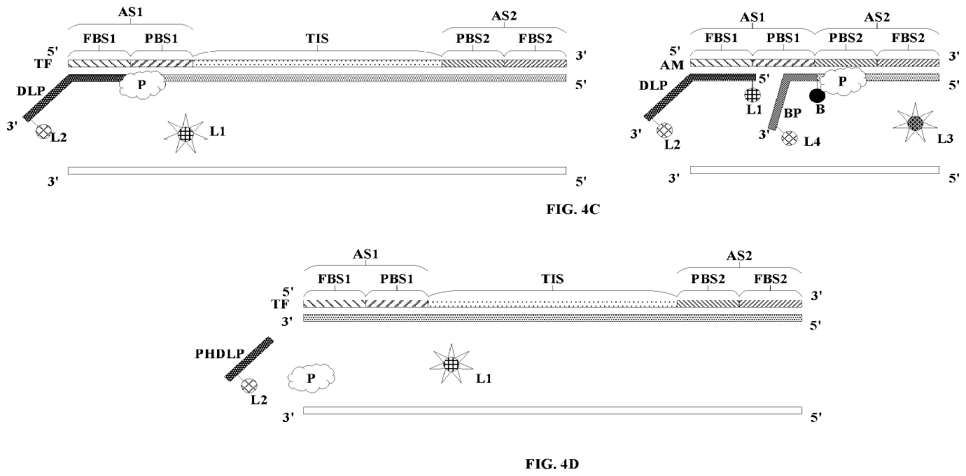

In some embodiments, each of the hybridizing oligonucleotides further includes a third labeled nucleic acid domain having a third label. FIGS. 4A to 4B illustrate several steps of a method of quantitating a plurality of nucleic acid molecules in some embodiments according to the present disclosure. Referring to FIG. 4B, in some embodiments, each of the hybridizing oligonucleotides BP further includes a third labeled nucleic acid domain having a third label L3, and a fourth labeled nucleic acid domain having a fourth label L4. The labels may be conjugated to the hybridizing oligonucleotide BP. Optionally, the third labeled nucleic acid domain is directly linked to the fourth labeled nucleic acid domain.

In some embodiments, the third labeled nucleic acid domain and the fourth labeled nucleic acid domain are two different domains selected from a second reporter nucleic acid domain and a second quencher nucleic acid domain, i.e., the hybridizing oligonucleotide in some embodiments includes a second reporter nucleic acid domain and a second quencher nucleic acid domain. Optionally, the third labeled nucleic acid domain is a second reporter nucleic acid domain; and the fourth labeled nucleic acid domain is a second quencher nucleic acid domain. Optionally, the third labeled nucleic acid domain is a second quencher nucleic acid domain; and the fourth labeled nucleic acid domain is a second reporter nucleic acid domain. Optionally, the third label L3 is a reporter moiety, and the fourth label L4 is a quencher moiety. Optionally, the third label L3 is a quencher moiety, and the fourth label L4 is a reporter moiety.

In some embodiments, subsequent to or during contacting the plurality of hybridizing oligonucleotides with the plurality of nucleic acid molecules, each of the hybridizing oligonucleotides hybridizes to the contiguous domain of the respective one of the plurality of adapter molecules, but not to the plurality of target nucleic acid fragments; hydrolyzation of each of the hybridizing oligonucleotides hybridized to the contiguous domain and hydrolyzation of each of the detectably-labeled probes hybridized to the first adapter sequence of each of the plurality of adapter molecules are blocked by a blocking moiety in the blocker nucleic acid domain; and the hybridizing oligonucleotides are partially hydrolyzed. Optionally, the method further includes detecting a second signal produced as a result of partially hydrolyzing the hybridizing oligonucleotides. Optionally, the first signal and the second signal are distinguishably detected. Optionally, the first signal is a first fluorescent signal, the second signal is a second fluorescent signal, and the signals detected upon the single cycle of extension reactions are fluorescent signals comprising the first fluorescent signal and the second fluorescent signal.

In some embodiments, the first signal and the second signal are two different signals having different detectable characteristics. Optionally, the first signal and the second signal are two different fluorescent signals having different detectable characteristics. Optionally, the second fluorescent signal has a different fluorescence color from that of the first fluorescent signal. Optionally, the first fluorescent signal and the second fluorescent signal are results of excitation by two different energy sources, for example, the first fluorescent signal is a result of excitation by a first energy source but the first fluorescent signal is zero or minimal upon excitation by a second energy source; and the second fluorescent signal is a result of excitation by the second energy source but the second fluorescent signal is zero or minimal upon excitation by the first energy source.

Referring to FIGS. 4B to 4D, in some embodiments, each of the hybridizing oligonucleotides BP includes a third labeled nucleic acid domain having a third label L3, and a fourth labeled nucleic acid domain having a fourth label L4. The third labeled nucleic acid domain is a second reporter domain, and the fourth labeled nucleic acid domain is a second quencher domain. The second reporter nucleic acid domain and the second quencher nucleic acid domain are linked by the blocker nucleic acid domain having the blocking moiety B. When the extension primer EP is extended to a hybridizing oligonucleotide BP, the polymerase P attacks the 5′-termini of the hybridizing oligonucleotide BP which is hybridized to the respective one of the adapter molecules AM, progressively degrading the hybridizing oligonucleotide BP, resulting in a progressive degradation of such hybridizing oligonucleotides BP. The hybridizing oligonucleotides BP are partially hydrolyzed to release at least one of the third label L3 or the fourth label L4. As a result, the third label L3 (e.g., a reporter moiety) and the fourth label L4 (e.g., a quencher moiety) are spaced apart from each other, e.g., by a distance greater than the minimum quenching distance, thereby allowing the reporter moiety to provide a fluorescence signal. When the progressive degradation continues to a blocking moiety B, the extension is blocked by the blocking moiety B.

In some embodiments, the method further includes calculating a number of the plurality of target nucleic acid fragments based on fluorescent signals detected. Because the hydrolyzation of each of the detectably-labeled probes hybridized to the first adapter sequence of a respective one of the plurality of adapter molecules is blocked by a blocking moiety in the blocker nucleic acid domain, the step of calculating the number of the plurality of target nucleic acid fragments is based on the first signal (e.g., a first fluorescent signal) detected.

In some embodiments, a total number of hydrolyzed detectably-labeled probes is substantially same as a total number of the plurality of target nucleic acid fragments prior to contacting the plurality of detectably-labeled probes and the plurality of extension primers with the plurality of nucleic acid molecules. In some embodiments, the total number of hydrolyzed detectably-labeled probes is correlated to an amplitude of the first signal; and the total number of the plurality of target nucleic acid fragments prior to contacting the plurality of detectably-labeled probes and the plurality of extension primers with the plurality of nucleic acid molecules is correlated to the amplitude of the first signal. Optionally, the first signal is a first fluorescent signal, and the signals detected upon the single cycle of extension reactions are fluorescent signals comprising the first fluorescent signal.

In some embodiments, a total number of partially hydrolyzed hybridizing oligonucleotides is substantially same as a total number of the plurality of adapter molecules prior to contacting the plurality of detectably-labeled probes and the plurality of extension primers with the plurality of nucleic acid molecules. In some embodiments, the total number of partially hydrolyzed hybridizing oligonucleotides is correlated to an amplitude of the second signal; and the total number of the plurality of adapter molecules prior to contacting the plurality of detectably-labeled probes and the plurality of extension primers with the plurality of nucleic acid molecules is correlated to the amplitude of the second signal. Optionally, the second signal is a second fluorescent signal.

One advantages of the present disclosure resides in that the method is capable of not only quantitating the plurality of target nucleic acid fragments in the library, but also quantitating the plurality of adapter molecules by detecting the first signal and the second signal. Optionally, the first signal is a first fluorescent signal, the second signal is a second fluorescent signal, and the signals detected upon the single cycle of extension reactions are fluorescent signals comprising the first fluorescent signal and the second fluorescent signal.

### Embodiment III

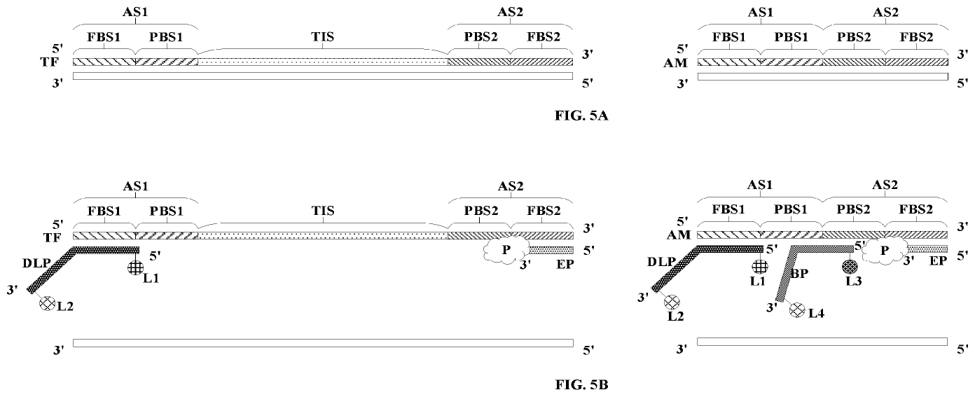

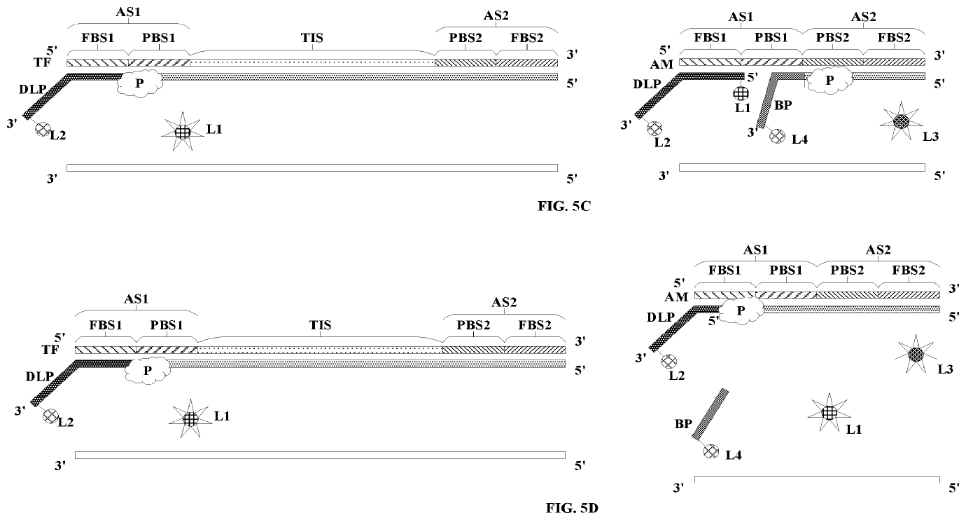

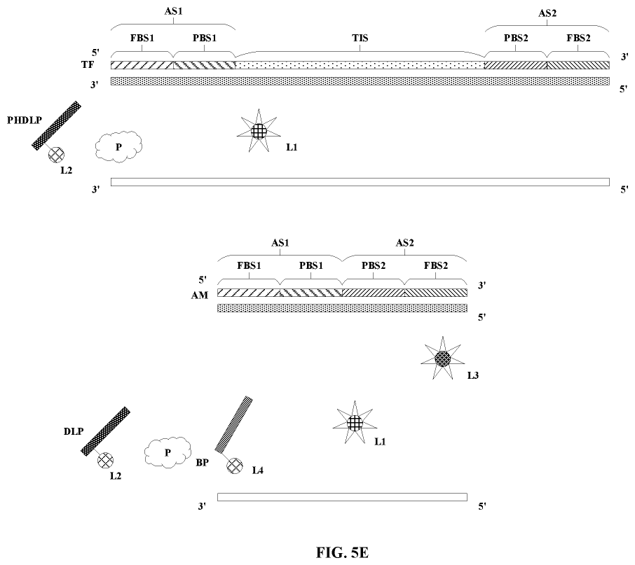

In some embodiments, each of the hybridizing oligonucleotides includes a third labeled nucleic acid domain having a third label, but does not include a blocker nucleic acid domain thus does not include a blocking moiety. FIGS. 5A to 5E illustrate several steps of a method of quantitating a plurality of nucleic acid molecules in some embodiments according to the present disclosure. Referring to FIG. 5B, in some embodiments, each of the hybridizing oligonucleotides BP includes a third labeled nucleic acid domain having a third label L3, and a fourth labeled nucleic acid domain having a fourth label L4. The labels may be conjugated to the hybridizing oligonucleotide BP. Optionally, the third labeled nucleic acid domain is directly linked to the fourth labeled nucleic acid domain.

In some embodiments, the third labeled nucleic acid domain and the fourth labeled nucleic acid domain are two different domains selected from a second reporter nucleic acid domain and a second quencher nucleic acid domain, i.e., the hybridizing oligonucleotide in some embodiments includes a second reporter nucleic acid domain and a second quencher nucleic acid domain. Optionally, the third labeled nucleic acid domain is a second reporter nucleic acid domain; and the fourth labeled nucleic acid domain is a second quencher nucleic acid domain. Optionally, the third labeled nucleic acid domain is a second quencher nucleic acid domain; and the fourth labeled nucleic acid domain is a second reporter nucleic acid domain. Optionally, the third label L3 is a reporter moiety, and the fourth label L4 is a quencher moiety. Optionally, the third label L3 is a quencher moiety, and the fourth label L4 is a reporter moiety.

In some embodiments, subsequent to or during contacting the plurality of hybridizing oligonucleotides with the plurality of nucleic acid molecules, each of the hybridizing oligonucleotides hybridizes to the contiguous domain of the respective one of the plurality of adapter molecules, but not to the plurality of target nucleic acid fragments. Because the hybridizing oligonucleotides do not include a blocker nucleic acid domain (i.e., a blocking moiety), hydrolyzation of each of the hybridizing oligonucleotides hybridized to the contiguous domain and hydrolyzation of each of the detectably-labeled probes hybridized to the first adapter sequence of each of the plurality of adapter molecules are not blocked. Hybridizing oligonucleotides and the detectably-labeled probes that hybridized to the plurality of adapter molecules are hydrolyzed.

In some embodiments, referring to FIGS. 5B to 5E, the method further includes producing an extension product of each of the plurality of adapter molecules AM by extending a respective one of the plurality of extension primers EP with the polymerase; and hydrolyzing each of the plurality of hybridizing oligonucleotides BP hybridized to the contiguous domain and hydrolyzing each of the plurality of detectably-labeled probes DLP hybridized to the respective one of the adapter molecule AMs, during producing an extension product of each of the plurality of adapter molecules AM.

In some embodiments, the method further includes detecting a second signal produced as a result of partially hydrolyzing the hybridizing oligonucleotides BP. In some embodiments, the first signal and the second signal are two different signals having different detectable characteristics. Optionally, the first fluorescent signal and the second fluorescent signal are two different fluorescent signals having different detectable characteristics. Optionally, the second fluorescent signal has a different fluorescence color from that of the first fluorescent signal. Optionally, the first fluorescent signal and the second fluorescent signal are results of excitation by two different energy sources, for example, the first fluorescent signal is a result of excitation by a first energy source but the first fluorescent signal is zero or minimal upon excitation by a second energy source; and the second fluorescent signal is a result of excitation by the second energy source but the second fluorescent signal is zero or minimal upon excitation by the first energy source.

Referring to FIGS. 5B to 5D, in some embodiments, each of the hybridizing oligonucleotides BP includes a third labeled nucleic acid domain having a third label L3, and a fourth labeled nucleic acid domain having a fourth label L4. The third labeled nucleic acid domain is a second reporter domain, and the fourth labeled nucleic acid domain is a second quencher domain. The second reporter nucleic acid domain and the second quencher nucleic acid domain are directly linked to each other without a blocker nucleic acid domain. When the extension primer EP is extended to a hybridizing oligonucleotide BP, the polymerase P attacks the 5′-termini of the hybridizing oligonucleotide BP which is hybridized to the respective one of the adapter molecules AM, progressively degrading the hybridizing oligonucleotide BP, resulting in a progressive degradation of such hybridizing oligonucleotides BP. The hybridizing oligonucleotides BP are hydrolyzed to release at least one of the third label L3 or the fourth label L4. As a result, the third label L3 (e.g., a reporter moiety) and the fourth label L4 (e.g., a quencher moiety) are spaced apart from each other, e.g., by a distance greater than the minimum quenching distance, thereby allowing the reporter moiety to provide a fluorescence signal.

Referring to FIGS. 5D to 5E, subsequent to hybridizing oligonucleotides BP being hydrolyzed, the partial extension product continues extending to the first labeled nucleic acid domain having the first label L1 of the detectably-labeled probe DLP, the polymerase P attacks the 5′-termini of the first labeled nucleic acid domain which is hybridized to the respective one of the adaptor molecules AM, progressively degrading the first labeled nucleic acid domain. As a result, the first label L1 is released from the detectably-labeled probe DLP. In one example, the first label L1 and the second label L2 constitute a reporter-quencher pair. In an intact detectably-labeled probe DLP, the first label L1 and the second label L2 are within a minimum quenching distance, and the energy released from an excited reported is quenched by the quencher. When the first label L1 is released from the detectably-labeled probe DLP, the first label L1 and the second label L2 are spaced apart from each other, e.g., by a distance greater than the minimum quenching distance, thereby allowing the reporter moiety (e.g., the first label L1) to provide a fluorescence signal.

In some embodiments, the method further includes detecting a first signal produced as a result of hydrolyzing the detectably-labeled probes DLP. The first signal is a result of hydrolyzing the detectably-labeled probes DLP hybridized to the plurality of target nucleic acid fragments TF and hydrolyzing the detectably-labeled probes DLP hybridized to the plurality of adaptor molecules AM. Optionally, the first signal is a first fluorescent signal, and the signals detected upon the single cycle of extension reactions are fluorescent signals comprising the first fluorescent signal.

In some embodiments, the method further includes detecting a second signal produced as a result of hydrolyzing the hybridizing oligonucleotide BP. The second signal is a result of hydrolyzing the hybridizing oligonucleotide BP hybridized to the plurality of adaptor molecules AM. Optionally, the first signal and the second signal are distinguishably detected. Optionally, the first signal is a first fluorescent signal, the second signal is a second fluorescent signal, and the signals detected upon the single cycle of extension reactions are fluorescent signals comprising the first fluorescent signal and the second fluorescent signal.

In some embodiments, the method further includes calculating a number of the plurality of target nucleic acid fragments based on fluorescent signals detected. Because the hydrolyzation of each of the detectably-labeled probes hybridized to the first adapter sequence of a respective one of the plurality of adapter molecules is not blocked by a blocking moiety, the step of calculating the number of the plurality of target nucleic acid fragments is based on a combination of the first signal and the second signal detected, e.g., based on a combination of the first fluorescent signal and the second fluorescent signal detected.

In some embodiments, a total number of hydrolyzed detectably-labeled probes is substantially same as a combination of a total number of the plurality of target nucleic acid fragments and a total number of the plurality of adaptor molecules prior to contacting the plurality of detectably-labeled probes and the plurality of extension primers with the plurality of nucleic acid molecules. In some embodiments, the total number of hydrolyzed detectably-labeled probes is correlated to a sum of an amplitude of the first signal and an amplitude of the second signal; and the total number of the plurality of target nucleic acid fragments prior to contacting the plurality of detectably-labeled probes and the plurality of extension primers with the plurality of nucleic acid molecules is correlated to a difference between the amplitude of the first signal and the amplitude of the second signal. Optionally, the first signal is a first fluorescent signal, the second signal is a second fluorescent signal, and the signals detected upon the single cycle of extension reactions are fluorescent signals comprising the first fluorescent signal and the second fluorescent signal.

In some embodiments, a total number of partially hydrolyzed hybridizing oligonucleotides is substantially same as a total number of the plurality of adapter molecules prior to contacting the plurality of detectably-labeled probes and the plurality of extension primers with the plurality of nucleic acid molecules. In some embodiments, the total number of partially hydrolyzed hybridizing oligonucleotides is correlated to an amplitude of the second signal; and the total number of the plurality of adapter molecules prior to contacting the plurality of detectably-labeled probes and the plurality of extension primers with the plurality of nucleic acid molecules is correlated to the amplitude of the second signal. Optionally, the second signal is a second fluorescent signal.

In some embodiments, the extension primers includes at least one of the plurality of nucleic acid molecules that non-specifically binds to a respective one of the plurality of target nucleic acid fragments. Optionally, all extension primers are multiple ones of the plurality of nucleic acid molecules in the library, each of which non-specifically binds to the respective one of the plurality of target nucleic acid fragments, e.g., no exogenous extension primers were added into the reaction. Optionally, the extension primers includes at least one of the plurality of target nucleic acid fragments that non-specifically binds to the respective one of the plurality of target nucleic acid fragments. Optionally, all extension primers are multiple ones of the plurality of target nucleic acid fragments that non-specifically binds to the respective one of the plurality of target nucleic acid fragments. Optionally, the endogenous extension primers includes at least one of the plurality of adaptor molecules that non-specifically binds to the respective one of the plurality of target nucleic acid fragments. Optionally, all extension primers are multiple ones of the plurality of adaptor molecules that non-specifically binds to the respective one of the plurality of target nucleic acid fragments.

One advantages of the present disclosure resides in that the method is capable of not only quantitating the plurality of target nucleic acid fragments in the library, but also quantitating the plurality of adapter molecules by detecting the first signal and the second signal, e.g., based on a combination of the first fluorescent signal and the second fluorescent signal detected.

In FIGS. 1A to 1D, FIGS. 2A to 2D, FIGS. 3A to 3B, FIGS. 4A to 4D, and FIGS. 5A to 5E, as an example, the detectably-labeled probe DLP includes a first labeled nucleic acid domain having a first label L1 and a second labeled nucleic acid domain having a second label L2; wherein the first labeled nucleic acid domain is a first reporter domain; the second labeled nucleic acid domain is a first quencher domain; the detectably-labeled probe DLP is hydrolyzed to release the first label L1 (e.g., a fluorophore).

In FIGS. 1A to 113, FIGS. 2A to 2D, FIGS. 3A to 3B, FIGS. 4A to 4D, and FIGS. 5A to 5E, as an example, the hybridizing oligonucleotide BP includes a third labeled nucleic acid domain having a third label L3 and a fourth labeled nucleic acid domain having a fourth label L4; wherein the third labeled nucleic acid domain is a second reporter domain; the fourth labeled nucleic acid domain is a second quencher domain; the hybridizing oligonucleotide BP is hydrolyzed to release the third label L3 (e.g., a fluorophore).

### Embodiment IV

In some embodiments, the detectably-labeled probe DLP includes a first labeled nucleic acid domain having a first label L1 and a second labeled nucleic acid domain having a second label L2; wherein the first labeled nucleic acid domain is a first reporter domain; the second labeled nucleic acid domain is a first quencher domain. Optionally, when the detectably-labeled probe DLP is partially hydrolyzed, the second label L2 (e.g., a quencher moiety) is released. Optionally, when the detectably-labeled probe DLP is hydrolyzed, the second label L2 (e.g., a quencher moiety) is released first.

### Embodiment V

In some embodiments, the hybridizing oligonucleotide BP includes a third labeled nucleic acid domain having a third label L3 and a fourth labeled nucleic acid domain having a fourth label L4; wherein the third labeled nucleic acid domain is a second reporter domain; the fourth labeled nucleic acid domain is a second quencher domain. Optionally, when the hybridizing oligonucleotide BP is partially hydrolyzed, the fourth label L4 (e.g., a quencher moiety) is released. Optionally, when the hybridizing oligonucleotide BP is hydrolyzed, the fourth label L4 (e.g., a quencher moiety) is released first.

### Embodiment VI

In some embodiments, the detectably-labeled probe DLP includes a first labeled nucleic acid domain having a first label L1 and a second labeled nucleic acid domain having a second label L2; wherein the first label L1 and the second label L2 are two spectrally similar or identical reporters. When the detectably-labeled probe DLP is hydrolyzed or partially hydrolyzed, the first label L1 and the second label L2 are spaced apart from each other, e.g., by a distance greater than a minimum quenching distance.

### Embodiment VII

In some embodiments, the hybridizing oligonucleotide BP includes a third labeled nucleic acid domain having a third label L3 and a fourth labeled nucleic acid domain having a fourth label L4; wherein the third label L3 and the fourth label L4 are two spectrally similar or identical reporters. When the hybridizing oligonucleotide BP is hydrolyzed or partially hydrolyzed, the third label L3 and the fourth label L4 are spaced apart from each other, e.g., by a distance greater than a minimum quenching distance.

### Embodiment VIII

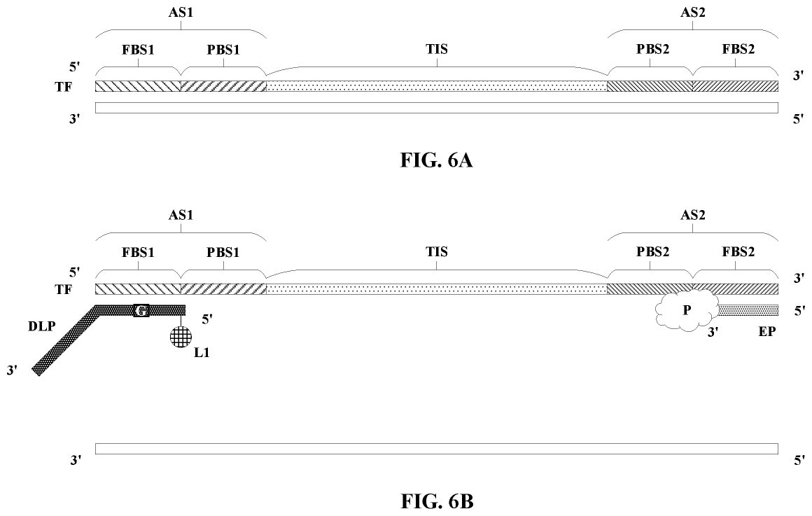

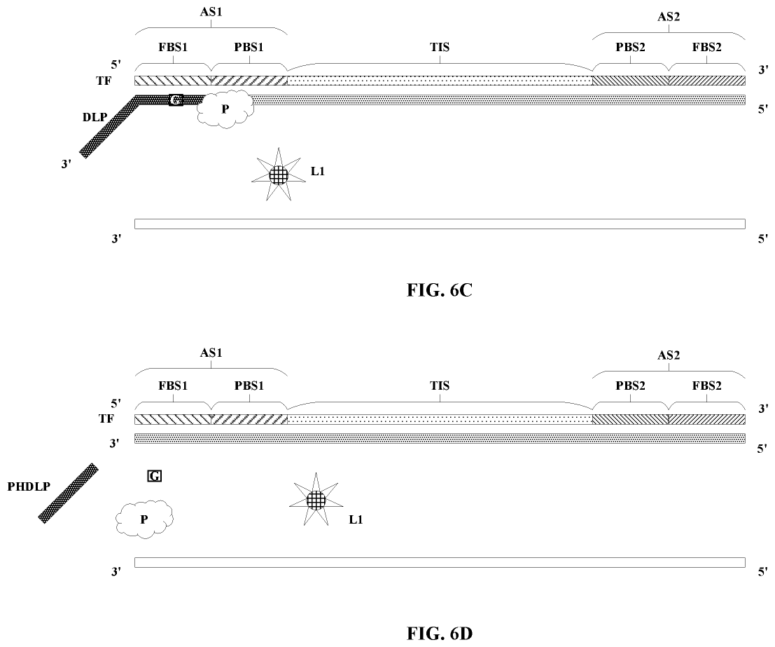

In some embodiments, the detectably-labeled probe DLP includes a first labeled nucleic acid domain having a first label L1, and a quenching nucleotide that quenches an energy from the first label L1 in an excited state, wherein the first label L1 is a reporter (e.g., a fluorophore). Examples of the quenching nucleotides include a guanidine. Optionally, when the detectably-labeled probe DLP is hydrolyzed or partially hydrolyzed, the quenching nucleotide is degraded first, thereby spacing apart the first label L1 and the quenching nucleotide. Optionally, when the detectably-labeled probe DLP is hydrolyzed or partially hydrolyzed, the first label L1 is degraded first, thereby spacing apart the first label L1 and the quenching nucleotide. Optionally, the detectably-labeled probe DLP does not include a second label L2. FIGS. 6A to 6D illustrate several steps of a method of quantitating a plurality of nucleic acid molecules in some embodiments according to the present disclosure. FIGS. 6A to 6D illustrate an embodiment in which the detectably-labeled probe DLP includes a first labeled nucleic acid domain having a first label L1, and a quenching nucleotide G.

### Embodiment IX

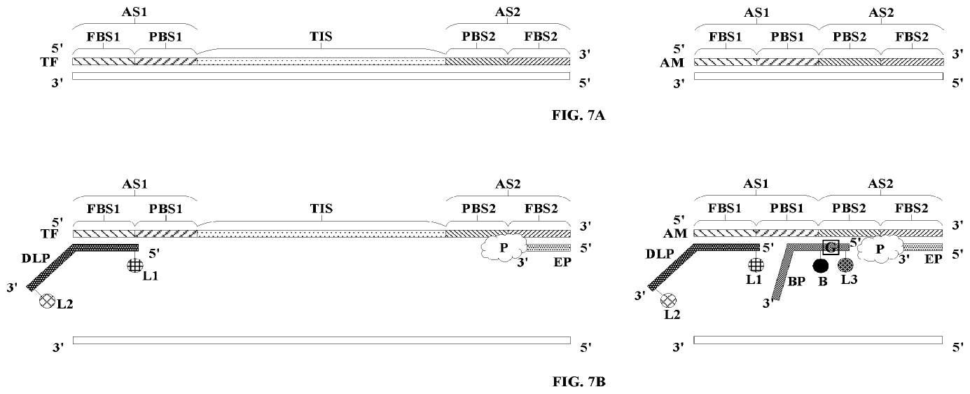

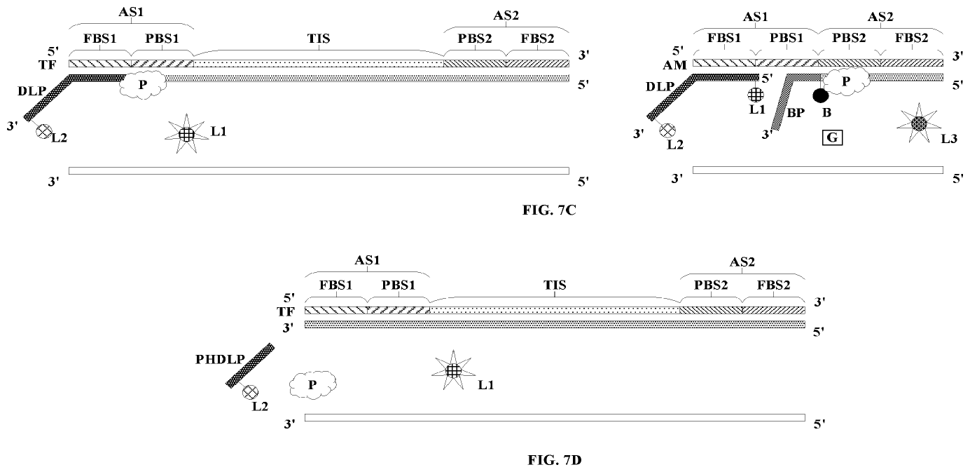

In some embodiments, the hybridizing oligonucleotide BP includes a third labeled nucleic acid domain having a third label L3, and a quenching nucleotide that quenches an energy from the third label L3 in an excited state, wherein the third label L3 is a reporter (e.g., a fluorophore). Examples of the quenching nucleotides include a guanidine. Optionally, when the hybridizing oligonucleotide BP is hydrolyzed or partially hydrolyzed, the quenching nucleotide is degraded first, thereby spacing apart the third label L3 and the quenching nucleotide. Optionally, when the hybridizing oligonucleotide BP is hydrolyzed or partially hydrolyzed, the third label L3 is degraded first, thereby spacing apart the third label L3 and the quenching nucleotide. Optionally, the hybridizing oligonucleotide BP does not include a fourth label L4. FIGS. 7A to 7D illustrate several steps of a method of quantitating a plurality of nucleic acid molecules in some embodiments according to the present disclosure. FIGS. 7A to 7D illustrate an embodiment in which the hybridizing oligonucleotide BP includes a third labeled nucleic acid domain having a third label L3, and a quenching nucleotide G.

In any one of the exemplary embodiments described herein (e.g., Embodiments I-IX), unlike the traditional library quantitation methods, the present method does not require template amplification. For example, the extension reaction according to the present disclosure is a non-amplification type extension reaction (e.g., a non-PCR-type reaction). In one example, the extension reaction according to the present disclosure does not involve successive cycles of amplification. In another example, the extension reaction according to the present disclosure can be characterized as a linear reaction, e.g., one complementary strand is produced per template molecule single-strand. In another example, the extension reaction according to the present disclosure on the single-stranded template molecule produces a second single strand which is opposite in sense to the single-strand of the template molecule. Accordingly, where the single stranded template molecule is a positive strand, the second strand synthesized in the extension reaction is a negative strand. In some embodiments, during the extension reactions according to the present disclosure, only negative strand(s) is produced, and no positive strand(s) is produced.

In any one of the exemplary embodiments described herein (e.g., Embodiments I-IX), unlike the traditional library quantitation methods, the present method does not require a measurement of a Cq value. The term “Cq value”, as used herein, refers to the cycle threshold in real time PCR, which is the number of cycles required for a fluorescent signal to cross a background level. Cq levels are inversely proportional to the amount of target nucleic acid in the sample, meaning that a lower Cq level indicates a higher amount of target nucleic acid template in the sample. In general, for a real time PCR assay undergoing 40 cycles of amplification, a Cq value of <29 is indicative of abundant target nucleic acid template in a sample, a Cq value of 30-37 is indicative of a moderate amount of target nucleic acid template, while a Cq value of 38-40 is indicative of a low amount of target nucleic acid template. Accordingly, in some embodiments, the step of calculating a number of the plurality of target nucleic acid fragments based on fluorescent signals detected is performed without relying on a Cq value.

In some embodiments, signals (e.g., fluorescent signals) detected upon the single cycle of extension reactions is relied on to calculate a number of the plurality of target nucleic acid fragments, i.e., to quantitate the library. For example, in some embodiments, calculating a number of the plurality of target nucleic acid fragments is exclusively based on signals (e.g., fluorescent signals) detected upon the single cycle of extension reactions. As used herein, the term “cycle” refers to the process which results in the production of a copy of a target nucleic acid. A cycle includes an annealing step and an extending step, and optionally further includes a denaturing step. A single cycle of extension reactions refers to a single run of an annealing step and an extending step without any additional cycle.

Accordingly, in some embodiments, the method for quantitating a plurality of nucleic acid molecules includes contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating a number of the plurality of target nucleic acid fragments based on signals (e.g., fluorescent signals) detected upon a single cycle of extension reactions.

Optionally, throughout the entire process of library quantitation, only a single cycle of extension reactions is performed. In one example, the method includes contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers in a single cycle of extension reactions with a polymerase; hydrolyzing each of the plurality of detectably-labeled probes hybridized to the respective one of the target nucleic acid fragments during extending the respective one of the plurality of extension primers with the polymerase in the single cycle of extension reactions; detecting a first signal produced as a result of hydrolyzing the plurality of detectably-labeled probes; and calculating a number of the plurality of target nucleic acid fragments based on signals (e.g., fluorescent signals) detected upon the single cycle of extension reactions. Optionally, the first signal is a first fluorescent signal, and the signals detected upon the single cycle of extension reactions are fluorescent signals comprising the first fluorescent signal.

Optionally, subsequent to initially contacting the plurality of detectably-labeled probes and the plurality of extension primers with the plurality of nucleic acid molecules, and prior to detecting the first signal, no additional cycle of extension reactions is performed other than the single cycle of extension reactions.

Optionally, a plurality of cycles of extension reactions may be performed prior to calculating the number of the plurality of target nucleic acid fragments based on signals (e.g., fluorescent signals) detected upon the single cycle of extension reactions. In one example, the library is prepared by amplifying target nucleic acid fragments in a sample, e.g., using extension primers in absence of detectably-labeled probes. In another example, a plurality runs of extension reactions may be performed subsequent to contacting the detectably-labeled probes and the extension primers with the plurality of nucleic acid molecules and prior to detecting the first signal produced as the result of hydrolyzing the detectably-labeled probes, as long as calculating the number of the plurality of target nucleic acid fragments is based on signals (e.g., fluorescent signals) detected upon the single cycle of extension reactions (e.g., the last cycle). Thus, in some embodiments, signals detected upon the single cycle of extension reactions includes signals produced as the result of hydrolyzing the detectably-labeled probes in the single cycle of extension reactions (e.g., the last cycle). In some embodiments, signals detected upon the single cycle of extension reactions includes a combination of signals produced as the result of hydrolyzing the detectably-labeled probes in the single cycle of extension reactions (e.g., the last cycle) and signals produced as the result of hydrolyzing the detectably-labeled probes in previous cycles; and calculating the number of the plurality of target nucleic acid fragments is based on the combination of signals.

In some embodiments, the plurality of target nucleic acid fragments are a plurality of target double-strand DNA fragments. Optionally, only one type of extension primers is used, for example, only one of a forward primer or a reverse primer is used. Optionally, only the type of primer that hybridizes to a same strand as the detectably-labeled probe is used in the present method, e.g., the extension primer and the detectably-labeled probe hybridize to a same strand of target nucleic acid fragment.

The hybridizing oligonucleotides having a blocker nucleic acid domain may be used in any library quantitation methods, including the traditional quantitation methods such as a qPCR library quantitation method. Accordingly, in some embodiments, the method for quantitating a plurality of nucleic acid molecules includes contacting a plurality of detectably-labeled probes, a plurality of extension primers, and a plurality of hybridizing oligonucleotides with the plurality of nucleic acid molecules, wherein each of the a plurality of detectably-labeled probes comprises a first labeled nucleic acid domain comprising a first label, and each of the a plurality of hybridizing oligonucleotides comprises a blocker nucleic acid domain; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; and detecting a fluorescent signal produced as a result of hydrolyzing the plurality of detectably-labeled probes.

In some embodiments, each of the plurality of hybridizing oligonucleotides comprises a sequence complementary to a contiguous domain of a respective one of the plurality of adapter molecules. Optionally, subsequent to or during contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments. Optionally, each of the plurality of target nucleic acid fragments comprises a first adapter sequence, a target insert sequence, and a second adapter sequence, the first adapter sequence is linked to the second adapter sequence through the target insert sequence. Optionally, each of the plurality of adapter molecules comprises a first adapter sequence directly linked to a second adapter sequence. Optionally, hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain

Optionally, the contiguous domain comprises a portion of the first adapter sequence and a portion of the second adapter sequence directly adjacent to each other.

In some embodiments, each of the plurality of hybridizing oligonucleotides further comprises a third labeled nucleic acid domain comprising a third label. Optionally, subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments. Optionally, hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain. Optionally, the plurality of hybridizing oligonucleotides are partially hydrolyzed. Optionally, the method further includes detecting a second signal produced as a result of partially hydrolyzing the plurality of hybridizing oligonucleotides. Optionally, the second signal is a second fluorescent signal.

In some embodiments, each of the plurality of hybridizing oligonucleotides further comprises a fourth labeled nucleic acid domain comprising a fourth label. Optionally, the third labeled nucleic acid domain is a second reporter domain. Optionally, the fourth labeled nucleic acid domain is a second quencher domain. Optionally, the second reporter domain and the second quencher domain are linked by the blocker nucleic acid domain. Optionally, the plurality of hybridizing oligonucleotides are partially hydrolyzed to release at least one of the third label or the fourth label.

In some embodiments, each of the plurality of hybridizing oligonucleotides further comprises a fourth labeled nucleic acid domain comprising a fourth label. Optionally, the third label and the fourth label are two spectrally similar or identical reporters. Optionally, the plurality of hybridizing oligonucleotides are partially hydrolyzed to release at least one of the third label or the fourth label.

In some embodiments, the third labeled nucleic acid domain is a third reporter domain. Optionally, each of the plurality of hybridizing oligonucleotides comprises a quenching nucleotide that quenches an energy from the third label in an excited state. Optionally, the plurality of hybridizing oligonucleotides are partially hydrolyzed to release at least one of the quenching nucleotide or the third label.

In another aspect, the present disclosure provides a method of quantitating a plurality of nucleic acid molecules comprising a plurality of target nucleic acid fragments. In some embodiments, the method of quantitating a plurality of nucleic acid molecules comprising a plurality of target nucleic acid fragments includes contacting a plurality of extension primers and a plurality of nucleic acid binding dye molecules with the plurality of nucleic acid molecules; producing an extension product of each of the plurality of target nucleic acid fragments by extending a respective one of the plurality of extension primers with a polymerase; subsequent to producing the extension product of each of the plurality of target nucleic acid fragments, measuring a signal produced by the plurality of nucleic acid binding dye molecules; and estimating an average size of the plurality of target nucleic acid fragments based on a number of the plurality of target nucleic acid fragments and the signal produced by the plurality of nucleic acid binding dye molecules.

The number of the plurality of target nucleic acid fragments may be calculated using various appropriate methods. In one example, the number of the plurality of target nucleic acid fragments are calculated based on the method described above. Accordingly, in some embodiments, the method further includes contacting a plurality of detectably-labeled probes and the plurality of extension primers with the plurality of nucleic acid molecules, wherein each of the plurality of detectably-labeled probes comprises a first labeled nucleic acid domain comprising a first label; producing the extension product of each of the plurality of target nucleic acid fragments by extending the respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; and calculating the number of the plurality of target nucleic acid fragments based on signals detected upon extension reactions. Other appropriate methods such a qPCR may be used for calculating the number of the plurality of target nucleic acid fragments.

Optionally, the signal produced by the plurality of nucleic acid binding dye molecules is measured after the single cycle of extension reactions is performed.

In some embodiments, the method of estimating the average size of target nucleic acid fragments includes determining a correlation factor. Optionally, the method includes contacting nucleic acid molecules in a respective one of the plurality of reference nucleic acid libraries with multiple nucleic acid binding dye molecules and multiple extension primers; producing an extension product of each of the nucleic acid molecules in the respective one of the plurality of reference nucleic acid libraries by extending a respective one of the multiple extension primers with a polymerase; and subsequent to producing the extension product of each of the nucleic acid molecules in the respective one of the plurality of reference nucleic acid libraries, measuring a reference signal produced by the multiple nucleic acid binding dye molecules in the respective one of the plurality of reference nucleic acid libraries. Optionally, the method further includes reiterating the above steps for a plurality of reference nucleic acid libraries; and determining a correlation factor using reference signals respectively measured in the plurality of reference nucleic acid libraries.

In one example, a correlation factor for the respective one of the plurality of reference nucleic acid libraries is determined using Equation (1):

$$Correlation factor = \frac{S}{average size \times N} ; \qquad ( 1 ) ;$$

wherein S stands for the signal produced by the plurality of nucleic acid binding dye molecules in the respective one of the plurality of reference nucleic acid libraries, and N stands for the number of nucleic acid molecules in the respective one of the plurality of reference nucleic acid libraries. For the reference nucleic acid libraries, the average size (e.g., in form of the number of base pairs) and N (e.g., in form of molarity) are known parameters, correlation factors for the reference nucleic acid libraries can be accordingly determined once the signal produced by the plurality of nucleic acid binding dye molecules is determined.

In another example, correlation factors are determined respectively for the plurality of reference nucleic acid libraries, and an average correlation factor is obtained as the correlation factor for determining the average size of an unknown library.

In some embodiments, the average size of the plurality of target nucleic acid fragments can be determined using Equation (2):

$$Average size = \frac{S}{correlation factor \times N} ; \qquad ( 2 )$$

wherein S stands for the signal produced by the plurality of nucleic acid binding dye molecules, and N stands for the number of the plurality of target nucleic acid fragments (e.g., in form of molarity). The correlation factor is determined using the plurality of reference nucleic acid libraries as described above (e.g., an average value of the correlation factors determined respectively for the plurality of reference nucleic acid libraries).

In some embodiments, the method further includes estimating a molarity of the plurality of target nucleic acid fragments. Optionally, the step of estimating the molarity of the plurality of target nucleic acid fragments includes determining a second correlation factor. Optionally, the method includes contacting multiple detectably-labeled probes and multiple extension primers with a respective one of the plurality of reference nucleic acid libraries, wherein each of the multiple detectably-labeled probes comprises a first labeled nucleic acid domain comprising a first label; producing an extension product of each of the nucleic acid molecules in the respective one of the plurality of reference nucleic acid libraries by extending a respective one of the multiple extension primers with a polymerase; hydrolyzing each of the multiple detectably-labeled probes hybridized to a respective one of the nucleic acid molecules in the respective one of the plurality of reference nucleic acid libraries during extending the respective one of the multiple extension primers with the polymerase; detecting a first reference signal produced as a result of hydrolyzing the plurality of detectably-labeled probes. Optionally, the method further includes reiterating the above steps for a plurality of reference nucleic acid libraries; and determining a second correlation factor using first reference signals (e.g., VIC fluorescence signals) respectively measured in the plurality of reference nucleic acid libraries.

In some embodiments, the method further includes plotting the first reference signal respectively measured in the plurality of reference nucleic acid libraries in a curve, and determining a slope and an intercept of the curve.

In some embodiments, a molarity of the plurality of target nucleic acid fragments can be determined using Equation (3):

Molarity=(first signal*slope)+intercept (3);

wherein the first signal is a signal produced as a result of hydrolyzing the plurality of detectably-labeled probes, as discussed above. The value of molarity may be used in calculating the average size of the plurality of target nucleic acid fragments using Equation (2).

In some embodiments, the method of estimating the average size of target nucleic acid fragments includes contacting the plurality of nucleic acid molecules with a plurality of nucleic acid binding dye molecules and a plurality of extension primers; measuring a third signal produced by the plurality of nucleic acid binding dye molecules; producing an extension product of each of the plurality of target nucleic acid fragments by extending a respective one of the plurality of extension primers with a polymerase; subsequent to producing the extension product of each of the plurality of target nucleic acid fragments, measuring a fourth signal produced by the plurality of nucleic acid binding dye molecules; and estimating a mass of the plurality of target nucleic acid fragments based on the third signal and the fourth signal.

In some embodiments, the method of estimating the average size of target nucleic acid fragments can be combined with the method of calculating the number of the plurality of target nucleic acid fragments. Optionally, the method of estimating the average size of target nucleic acid fragments and the method of calculating the number of the plurality of target nucleic acid fragments can be performed based on extension reactions, as described above in connection with the method of calculating the number of the plurality of target nucleic acid fragments. Accordingly, in some embodiments, the method of estimating the average size of target nucleic acid fragments includes contacting a plurality of detectably-labeled probes and a plurality of extension primers with the plurality of nucleic acid molecules, wherein each of the plurality of detectably-labeled probes comprises a first labeled nucleic acid domain comprising a first label; contacting the plurality of nucleic acid molecules with a plurality of nucleic acid binding dye molecules; measuring a third signal produced by the plurality of nucleic acid binding dye molecules; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; detecting a first signal produced as a result of hydrolyzing the plurality of detectably-labeled probes; calculating a number of the plurality of target nucleic acid fragments based on signals detected upon extension reactions; measuring a fourth signal produced by the plurality of nucleic acid binding dye molecules; and estimating a mass of the plurality of target nucleic acid fragments based on the third signal and the fourth signal.

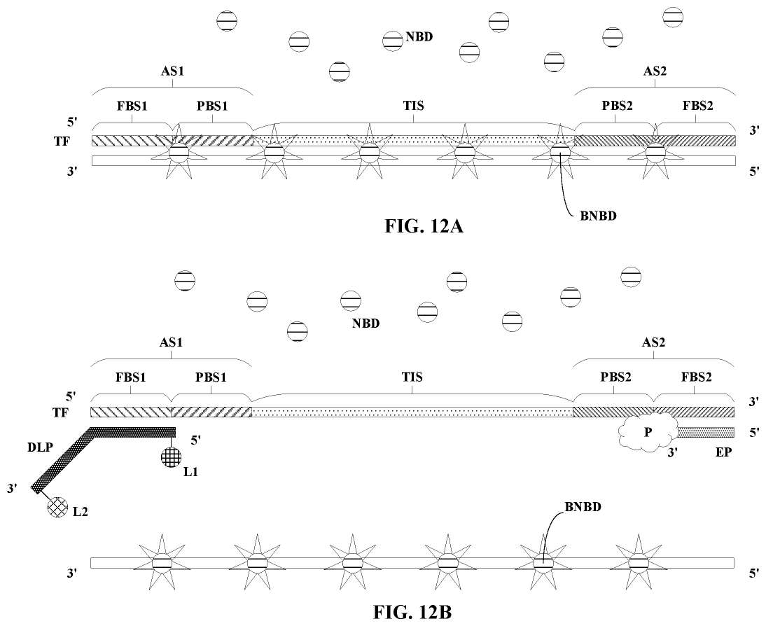

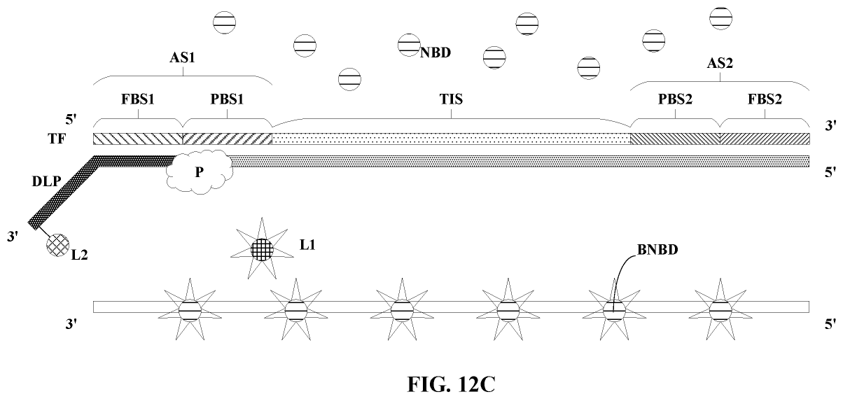

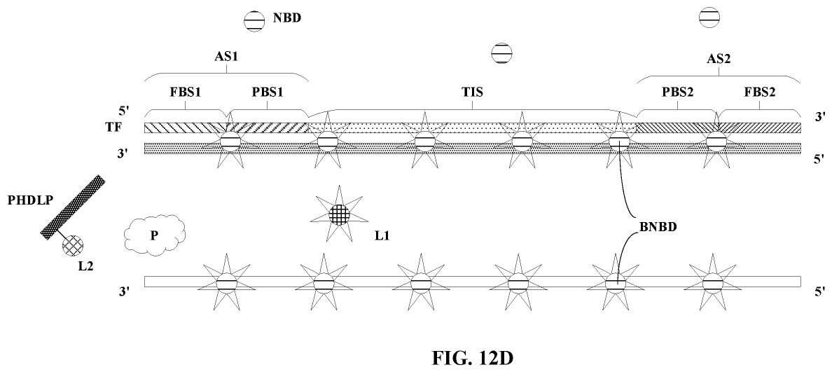

In some embodiments, the method of estimating the average size of target nucleic acid fragments further includes estimating an average size of the plurality of target nucleic acid fragments based on the number of the plurality of target nucleic acid fragments and the mass of the plurality of target nucleic acid fragments. FIGS. 12A to 12D illustrate several steps of a method of estimating an average size of a plurality of target nucleic acid fragments in some embodiments according to the present disclosure. FIGS. 12A to 12D illustrate an example in which the method of estimating the average size of target nucleic acid fragments is performed in combination with the method of calculating the number of the plurality of target nucleic acid fragments. In this example, the method of calculating the number of the plurality of target nucleic acid fragments is performed according to the steps depicted in Embodiment I described above. However, the method of estimating the average size of target nucleic acid fragments may be performed independently, or in combination with various other embodiments (e.g., Embodiments II to IX described above).

Referring to FIG. 12A, the method includes a step of contacting the plurality of nucleic acid molecules with a plurality of nucleic acid binding dye molecules NBD and extension primers EP. Optionally, as depicted in FIG. 12A, the method includes contacting the plurality of nucleic acid molecules with the plurality of nucleic acid binding dye molecules NBD, detectably-labeled probes DLP, and extension primers EP. Referring to FIG. 12A and FIG. 12B, the plurality of nucleic acid binding dye molecules NBD, detectably-labeled probes DLP, and extension primers EP are added to a solution containing the plurality of nucleic acid molecules. The plurality of nucleic acid molecules include the plurality of target nucleic acid fragments TF.

Referring to FIG. 12A, when the nucleic acid binding dye molecule binds to the plurality of target nucleic acid fragments TF (i.e., a bound nucleic acid binding dye molecule BNBD), the bound nucleic acid binding dye molecule BNBD produces a signal (e.g., a fluorescence signal). Optionally, the nucleic acid binding dye molecules produces a stronger signal when it is bound to the target nucleic acid fragments as compared to the signal it produces when it is in an unbound state. In some embodiments, an amount of nucleic acid binding dye molecules bound to nucleic acid molecules is correlated to a mass of the nucleic acid molecules. Thus, by measuring a signal produced by the nucleic acid binding dye molecules that are bound to the target nucleic acid fragments, a mass of the target nucleic acid fragments can be determined.

Referring to FIG. 12B and FIG. 12C, an extension primer EP, in the presence of a polymerase P and under an extension condition, is extended to produce an extension product of the respective one of the plurality of target nucleic acid fragments TF. Referring to FIG. 12D, the extension primer EP continues extending to hydrolyze the hybridized portion of the detectably-labeled probe DLP hybridized to the respective one of the target nucleic acid fragments TF, releasing the non-hybridized portion of the detectably-labeled probe.

In some embodiments, the method includes, prior to producing the extension product of each of the plurality of target nucleic acid fragments (see, e.g., FIG. 12A), measuring a third signal produced by the plurality of nucleic acid binding dye molecules. Optionally, the third signal includes a signal produced by bound nucleic acid binding dye molecules BNBD (for example, the signal produced by the unbound nucleic acid binding dye molecules is negligible). Optionally, the third signal includes a combination of a signal produced by bound nucleic acid binding dye molecules BNBD and a signal produced by unbound nucleic acid binding dye molecules.

In some embodiments, the method includes, subsequent to producing the extension product of each of the plurality of target nucleic acid fragments (see, e.g., FIG. 12D), measuring a fourth signal produced by the plurality of nucleic acid binding dye molecules. Optionally, the fourth signal includes a signal produced by bound nucleic acid binding dye molecules BNBD (for example, the signal produced by the unbound nucleic acid binding dye molecules is negligible). Optionally, the fourth signal includes a combination of a signal produced by bound nucleic acid binding dye molecules BNBD and a signal produced by unbound nucleic acid binding dye molecules.

The mass of the plurality of target nucleic acid fragments can be estimated based on the third signal and the fourth signal. In one example, the mass of the plurality of target nucleic acid fragments can be estimated based on the difference between the third signal and the fourth signal. As shown in FIG. 12A, in one example, prior to producing the extension product, six nucleic acid binding dye molecules bind to a double strand nucleic acid molecule. As shown in FIG. 12D, subsequent to producing the extension product, a double strand nucleic acid and a single strand extension product are present in the sample. In one example, six nucleic acid binding dye molecules bind to the double strand nucleic acid molecule, and six nucleic acid binding dye molecules bind to the single strand extension product. A difference between the third signal and the fourth signal is correlated to a mass of the single strand extension product (in single strand format), or in turn correlated to a mass of the double strand nucleic acid molecule (in double stand format).

The number of the plurality of target nucleic acid fragments can be determined, as discussed above, according to the method described in the present disclosure (for example, Embodiments I to IX). The average size of the plurality of target nucleic acid fragments can be determined based on the mass of the plurality of target nucleic acid fragments and the number of the plurality of target nucleic acid fragments.

In some embodiments, the fourth signal is measured after a single cycle of extension reactions is performed subsequent to measurement of the third signal. Optionally, the single cycle of extension reactions is the single cycle of extension reactions upon which signals are detected for calculating the number of the plurality of target nucleic acid fragments.

In some embodiments, the fourth signal is measured after a plurality of cycles of extension reactions are performed subsequent to measurement of the third signal. With each cycle of extension reactions, additional single strand extend products are produced. In one example, the fourth signal is measured after five cycles of extension reactions are performed subsequent to measurement of the third signal. At the time of measuring the fourth signal, the sample contains 1× double strand nucleic acid molecules, and 5× single strand extension products. A difference between the third signal and the fourth signal is correlated to a mass of the 5× single strand extension products. A mass of the plurality of target nucleic acid fragments can be determined based the difference between the third signal and the fourth signal.

In some embodiments, the plurality of nucleic acid binding dye molecules are fluorescence dye molecules. Optionally, the third signal and the fourth signal are respectively fluorescence signals. Optionally, the first signal, the second signal, and a signal produced by the plurality of nucleic acid binding dye molecules can be distinguishably detected. For example, the first signal, the second signal, and the signal produced by the plurality of nucleic acid binding dye molecules are of different wavelengths that allow them to be distinguishably detected.

In another aspect, the present disclosure provides a method of sequencing a nucleic acid sample. One advantages of the present method resides in that the library used for quantitating the plurality of target nucleic acid fragments may be reused for sequencing. Accordingly, in some embodiments, the method of sequencing the nucleic acid sample includes generating a plurality of nucleic acid molecules comprising a plurality of target nucleic acid fragments using a nucleic acid sample; contacting a plurality of detectably-labeled probes and a plurality of extension primers with the plurality of nucleic acid molecules, wherein each of the plurality of detectably-labeled probes comprises a first labeled nucleic acid domain comprising a first label; producing an extension product of each of the plurality of target nucleic acid fragments by extending a respective one of the plurality of extension primers in a single cycle of extension reactions with a polymerase; hydrolyzing each of the plurality of detectably-labeled probes hybridized to the respective one of the target nucleic acid fragments during extending the respective one of the plurality of extension primers with the polymerase in the single cycle of extension reactions; detecting a first signal produced as a result of hydrolyzing the plurality of detectably-labeled probes; calculating a number of the plurality of target nucleic acid fragments based on signals (e.g., fluorescent signals) detected upon the single cycle of extension reactions; diluting the plurality of target nucleic acid fragments to a predetermined concentration; and sequencing at least one portion of the plurality of target nucleic acid fragments. Optionally, the first signal is a first fluorescent signal, and the signals detected upon the single cycle of extension reactions are fluorescent signals comprising the first fluorescent signal.

In another aspect, the present disclosure provides a mixture. In some embodiments, the mixture includes a plurality of nucleic acid molecules comprising a plurality of target nucleic acid fragments; a plurality of detectably-labeled probes. Optionally, each of the plurality of detectably-labeled probes comprises a first labeled nucleic acid domain comprising a first label. Optionally, each of the plurality of target nucleic acid fragments comprises a first adapter sequence, a target insert sequence, and a second adapter sequence. Optionally, the first adapter sequence is linked to the second adapter sequence through the target insert sequence. Optionally, at least a portion of each of the plurality of detectably-labeled probes is complementary to at least a portion of the first adapter sequence.

In some embodiments, the mixture further includes a plurality of hybridized adapter molecules. Optionally, each of the plurality of hybridized adapter molecules includes the first adapter sequence; the second adapter sequence; and a partially hydrolyzed hybridizing oligonucleotide, at least a portion of which is hybridized to a domain of a respective one of a plurality of adapter molecules. Optionally, the first adapter sequence is directly linked to the second adapter sequence. Optionally, the domain comprises a portion of the first adapter sequence and a portion of the second adapter sequence directly adjacent to each other.

In some embodiments, each of the plurality of hybridized adapter molecules further includes one of the plurality of detectably-labeled probes, at least a portion of which is hybridized to at least a portion of the first adapter sequence; and one of a plurality of extension primers, at least a portion of which is hybridized to at least a portion of the at least a portion of the second adapter sequence.

In some embodiments, each of the plurality of detectably-labeled probes further includes a second labeled nucleic acid domain comprising a second label. Optionally, the first labeled nucleic acid domain is a first reporter domain. Optionally, the second labeled nucleic acid domain is a first quencher domain.

In some embodiments, each of the plurality of detectably-labeled probes further includes a second labeled nucleic acid domain comprising a second label. Optionally, the first label and the second label are two spectrally similar or identical reporters.

In some embodiments, the first labeled nucleic acid domain is a first reporter domain. Optionally, each of the plurality of detectably-labeled probes comprises a quenching nucleotide that quenches an energy from the first label in an excited state.

In some embodiments, the first adapter sequence includes a first flow cell binding sequence at its 5′ end. Optionally, at least a portion of each of the plurality of detectably-labeled probes is complementary to at least a portion of the first flow cell binding sequence.

In some embodiments, the mixture further includes a plurality of extension primers. Optionally, at least a portion of each of the plurality of extension primers is complementary to at least a portion of the second adapter sequence.

In some embodiments, the second adapter sequence includes a second flow cell binding sequence at its 3′ end. Optionally, at least a portion of each of the plurality of extension primers is complementary to at least a portion of the second flow cell binding sequence.

In some embodiments, the mixture further includes a plurality of hybridizing oligonucleotides. Optionally, each of the plurality of hybridizing oligonucleotides comprises a blocker nucleic acid domain.

In some embodiments, each of the plurality of hybridizing oligonucleotides further includes a third labeled nucleic acid domain comprising a third label.

In some embodiments, each of the plurality of hybridizing oligonucleotides further includes a fourth labeled nucleic acid domain comprising a fourth label. Optionally, the third labeled nucleic acid domain is a second reporter domain. Optionally, the fourth labeled nucleic acid domain is a second quencher domain. Optionally, the second reporter domain and the second quencher domain are linked by the blocker nucleic acid domain.

In some embodiments, each of the plurality of hybridizing oligonucleotides further includes a fourth labeled nucleic acid domain comprising a fourth label. Optionally, the third label and the fourth label are two spectrally similar or identical reporters.

In some embodiments, the third labeled nucleic acid domain is a third reporter domain. Optionally, each of the plurality of hybridizing oligonucleotides includes a quenching nucleotide that quenches an energy from the third label in an excited state.

In some embodiments, the mixture further includes a plurality of nucleic acid binding dye molecules.

In another aspect, the present disclosure further provides a kit for quantitating a plurality of nucleic acid molecules according to any one the methods described herein. Typically, the kit is compartmentalized for ease of use and contains at least one container providing a probe of the present invention as described herein. One or more additional containers providing additional reagent(s) can also be included. Such additional containers can include any reagents or other elements for use in primer extension procedures in accordance with the methods described above, including reagents for use in, e.g., nucleic acid amplification procedures, DNA sequencing procedures, or DNA labeling procedures.

In some embodiments, the kit includes a container providing a plurality of detectably-labeled probes. In some embodiments, the kit further includes a container providing a plurality of extension primers. Optionally, the kit includes a container providing a mixture comprising a plurality of detectably-labeled probes and a plurality of extension primers.

In some embodiments, the kit further includes instructions directing a user to perform any of the methods described in the present disclosure. In an exemplary embodiments, the kit includes instructions directing a user to (1) producing an extension product of each of the plurality of target nucleic acid fragments by extending a respective one of the plurality of extension primers with a polymerase; and (2) calculating a number of the plurality of target nucleic acid fragments based on signals (e.g., fluorescent signals) detected upon a single cycle of extension reactions. In another exemplary embodiments, the kit includes instructions directing a user to (1) producing an extension product of each of the plurality of target nucleic acid fragments by extending a respective one of the plurality of extension primers with a polymerase in a single cycle of extension reactions; and (2) calculating a number of the plurality of target nucleic acid fragments based on signals (e.g., fluorescent signals) detected upon the single cycle of extension reactions. In another exemplary embodiments, the kit includes instructions directing a user to (1) contacting a plurality of detectably-labeled probes and a plurality of extension primers with the plurality of nucleic acid molecules, wherein each of the plurality of detectably-labeled probes comprises a first labeled nucleic acid domain comprising a first label; (2) producing an extension product of each of the plurality of target nucleic acid fragments by extending a respective one of the plurality of extension primers in a single cycle of extension reactions with a polymerase; (3) hydrolyzing each of the plurality of detectably-labeled probes hybridized to the respective one of the target nucleic acid fragments during extending the respective one of the plurality of extension primers with the polymerase in the single cycle of extension reactions; (4) detecting a first signal produced as a result of hydrolyzing the plurality of detectably-labeled probes; and (5) calculating a number of the plurality of target nucleic acid fragments based on signals (e.g., fluorescent signals) detected upon the single cycle of extension reactions. Optionally, the first signal is a first fluorescent signal, and the signals detected upon the single cycle of extension reactions are fluorescent signals comprising the first fluorescent signal.

In some embodiments, the kit includes instructions directing a user to (1) contacting a plurality of detectably-labeled probes, a plurality of extension primers, and a plurality of hybridizing oligonucleotides with the plurality of nucleic acid molecules, wherein each of the a plurality of detectably-labeled probes comprises a first labeled nucleic acid domain comprising a first label, and each of the a plurality of hybridizing oligonucleotides comprises a blocker nucleic acid domain; producing an extension product of each of the plurality of target nucleic acid fragments by extending a respective one of the plurality of extension primers with a polymerase; hydrolyzing each of the plurality of detectably-labeled probes hybridized to the respective one of the target nucleic acid fragments during extending the respective one of the plurality of extension primers with the polymerase; and detecting a fluorescent signal produced as a result of hydrolyzing the plurality of detectably-labeled probes.

In some embodiments, each of the plurality of detectably-labeled probes comprises a first labeled nucleic acid domain comprising a first label; at least a portion of each of plurality of the detectably-labeled probes is complementary to at least a portion of the first adapter sequence; and at least a portion of each of the plurality of extension primers is complementary to at least a portion of the second adapter sequence.

In some embodiments, each of the plurality of detectably-labeled probes further comprises a second labeled nucleic acid domain comprising a second label; the first labeled nucleic acid domain is a first reporter domain; and the second labeled nucleic acid domain is a first quencher domain.

In some embodiments, each of the plurality of detectably-labeled probes further comprises a second labeled nucleic acid domain comprising a second label; the first label and the second label are two spectrally similar or identical reporters.

In some embodiments, the first labeled nucleic acid domain is a first reporter domain; and each of the plurality of detectably-labeled probes comprises a quenching nucleotide that quenches an energy from the first label in an excited state.

In some embodiments, the first adapter sequence comprises a first flow cell binding sequence at its 5′ end; at least a portion of each of the plurality of detectably-labeled probes is complementary to at least a portion of the first flow cell binding sequence.

In some embodiments, the second adapter sequence comprises a second flow cell binding sequence at its 3′ end; and at least a portion of each of the plurality of extension primers is complementary to at least a portion of the second flow cell binding sequence.

In some embodiments, the kit further includes a container providing a plurality of hybridizing oligonucleotides. Optionally, each of the plurality of hybridizing oligonucleotides comprises a blocker nucleic acid domain.

In some embodiments, each of the plurality of hybridizing oligonucleotides further comprises a third labeled nucleic acid domain comprising a third label.

In some embodiments, each of the plurality of hybridizing oligonucleotides further comprises a fourth labeled nucleic acid domain comprising a fourth label; the third labeled nucleic acid domain is a second reporter domain; the fourth labeled nucleic acid domain is a second quencher domain; and the second reporter domain and the second quencher domain are linked by the blocker nucleic acid domain.

In some embodiments, each of the plurality of hybridizing oligonucleotides further comprises a fourth labeled nucleic acid domain comprising a fourth label; the third label and the fourth label are two spectrally similar or identical reporters.

In some embodiments, the third labeled nucleic acid domain is a third reporter domain; and each of the plurality of hybridizing oligonucleotides comprises a quenching nucleotide that quenches an energy from the third label in an excited state.

In some embodiments, the kit further includes a container providing a polymerase.

In some embodiments, the kit further includes a plurality of nucleic acid binding dye molecules.

In still other, non-mutually exclusive embodiments, the kit includes one or more containers providing a buffer suitable for a primer extension reaction.

In other, non-mutually exclusive variations, the kit includes one or more containers providing free nucleotides (conventional and/or unconventional).

The following examples, references, and figures are provided to aid the understanding of the present invention, the true scope of which is set forth in the appended claims. It is understood that modifications can be made in the procedures set forth without departing from the spirit of the invention.

### EXAMPLES

### Example I. Materials

The following detectably-labeled probes were used in various examples described in this section.

(SEQ ID NO: 8)
/5Cy5/GTGTAGATCTCGGTGGTCGCCGTATCATT/3IAbRQSp/
(SEQ ID NO: 9)
/5Cy5/GTGTAGATC/iSuper-dT/CGGTGGTCGCCGTATCATT/
3IAbRQSp/
(SEQ ID NO: 10)
/5Cy5/GTGTAGATC/iSuper-dT/CGGTGG/iSuper-
dT/CGCCGTATCATT/3IAbRQSp/
(SEQ ID NO: 11)
/56-FAM/TGTAGATCT/ZEN/CGGTGGTCGCCGTATCATT/
3IABkFQ/
(SEQ ID NO: 12)
/56-FAM/TGTAGATCTCGGTGGTCGCCGTATCATTATCTACA/
3IABkFQ/
(SEQ ID NO: 13)
/56-FAM/TGTAGATCTCGGTGGTCGCCGTATCATT/3IABkFQ/
(SEQ ID NO: 14)
/56-FAM/TGTAGATCT/ZEN/CGGTGGTCGCCGTATCATTATCTACA/
3IABkFQ/
(SEQ ID NO: 15)
/56-FAM/AACTCCAGTCACATCTCGTATGCCGTCTTCTGCTTG/
3IABkFQ/
(SEQ ID NO: 16)
/56-FAM/CAGTCACATCTCGTATGCCGTCTTCTGCTTG/
3IABkFQ/
(SEQ ID NO: 17)
/56-FAM/ATCTCGTATGCCGTCTTCTGCTTG/3IABkFQ/
(SEQ ID NO: 18)
/56-FAM/CTCGTATGCCGTCTTCTGCTT/3IABkFQ/
(SEQ ID NO: 19)
/56-FAM/GTATGCCGTCTTCTGC/3IABkFQ/

/5Cy5/represents 5′ Cy5 (cyanine) fluorophore.

/3IAbRQSp/represents 3′ Iowa Black® RQ terminal quencher, made by Integrated DNA Technologies.

/iSuper-dT/represents 5-hydroxybutynl-2′-deoxyuridine, made by Integrated DNA Technologies.

/56-FAM/represents 5′ 6-FAM (fluorescein) fluorophore.

/ZEN/represents an internal ‘ZEN’ fluorescence quencher, made by Integrated DNA Technologies.

/3IABkFQ/represents 3′ Iowa Black® FQ terminal quencher, made by Integrated DNA Technologies.

The following extension primers were used in various examples described in this section.

(SEQ ID NO: 20)
5′-CAAGCAGAAGACGGCATACGAGAT-3′
(SEQ ID NO: 21)
5′-CAAGCAGAAGACGGCATACGAG-3′
(SEQ ID NO: 22)
5′-AAGCAGAAGACGGCATACGA-3′
(SEQ ID NO: 23)
5′-CAAGCAGAAGACGGCATAC-3′
(SEQ ID NO: 24)
5′-AATGATACGGCGACCACCGAGATCT-3′
(SEQ ID NO: 25)
5′-GATACGGCGACCACCGAGATCT-3′
(SEQ ID NO: 26)
5′-GATACGGCGACCACCGAG-3′
The following hybridizing oligonucleotides were
used in various examples described in this
section.
(SEQ ID NO: 27)
5′-AC*G*C*TCTTCCGATCTAGATCGGAAGAGCACA-3′
(SEQ ID NO: 28)
5′-CG*C*T*CTTCCGATCTAGATCGGAAGAGCA-3′
(SEQ ID NO: 29)
5′-CG*C*TCTTCCGATCTAGATCGGAAGAGCA-3′
(SEQ ID NO: 30)
5′-CG*A*C*GCTCTTCCGATCTAGATCGGAAGAGCACACG-3′
(SEQ ID NO: 31)
5′-CG*A*C*GGTCTTCCGATCTAGATCCGAAGAGCACACG-3′

Symbol * indicates a phosphorothioated nucleotide.

The following target nucleic acid fragments were used in various examples described in this section.

(SEQ ID NO: 32)
5′-AATGATACGGCGACCACCGAGATCTACACACACTCTTTCCCTACACG
ACGCTCTTCCGATCTAGATCGGAAGAGCACACGTCTGAACTCCAGTCACA
TCTCGTATGCCGTCTTCTGCTTG-3′
(SEQ ID NO: 33)
5′-AATGATACGGCGACCACCGAGATCTACACACACTCTTTCCCTTGAAC
TCCAGTCACATCTCGTATGCCGTCTTCTGCTTG-3′
(SEQ ID NO: 34)
5′-AATGATACGGCGACCACCGAGATCTACACACACTCTTTCCCTACACG
ACGCTCTTCCGATCTNNNNNNNNNNNNNNNNNNNNNNNNNNNNNNAGATC
GGAAGAGCACACGTCTGAACTCCAGTCACATCTCGTATGCCGTCTTCTGC
TTG-3′

The following complementary strands of several target nucleic acid fragments were used in various examples described in this section.

(SEQ ID NO: 35)
5′-GTTCGTCTTCTGCCGTATGCTCTACACTGACCTCAAGTCTGCACACG
AGAAGGCTAGATCTAGCCTTCTCGCAGCACATCCCTTTCTCACATCTAGA
GCCACCAGCGGCATAGTAA-3′
(SEQ ID NO: 36)
5′-GTTCGTCTTCTGCCGTATGCTCTACACTGACCTCAAGTCTGCACACG
ATCTAGCCTTCTCGCAGCACATCCCTTTCTCACATCTAGAGCCACCAGCG
GCATAGTAA-3′
(SEQ ID NO: 37)
5′-GTTCGTCTTCTGCCGTATGCTCTACACTGACCTCAAGTCTGCAGCAC
ATCCCTTTCTCACATCTAGAGCCACCAGCGGCATAGTAA-3′

### Example II. Reaction Sensitivity and Template Titration in Reactions

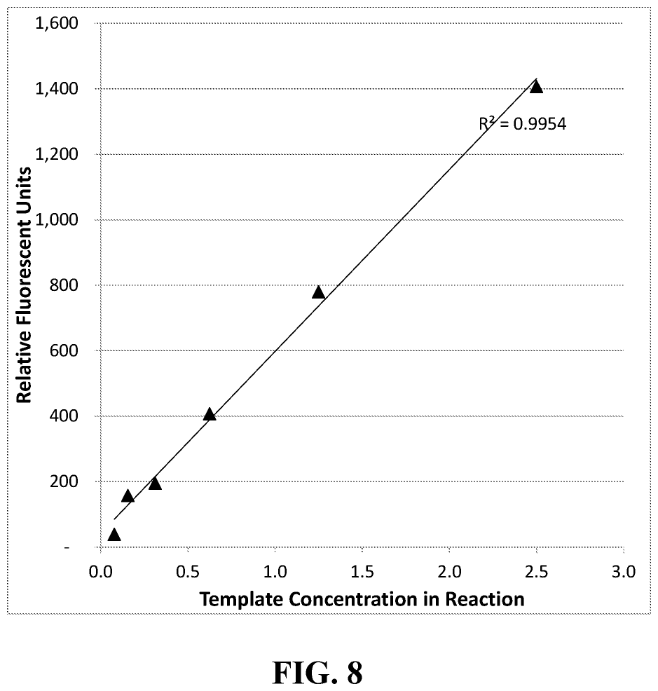

A library having oligonucleotides (SEQ ID NO. 32) containing SEQ ID NO. 1 on its 5′ end and SEQ ID NO. 4 on its 3′ end was serially diluted and put into reactions to get final concentrations from 0.08 nM to 2.5 nM as shown on the X-axis in FIG. 8. DNA polymerase with was added in combination with an extension primer (SEQ ID NO. 20) and a detectably-labeled probe (SEQ ID NO. 13). Incubation was done at 95 degrees for 60 seconds, 60 degrees for 60 seconds, and 24 degrees for 10 seconds followed by reading of fluorescence. Incubation and reading was performed on a QuantStudio 6 Pro (ThermoFisher, Catalog no. A43180) and plotted as Relative Fluorescent units after normalization using a passive reference dye 6-carboxy-X-rhodamine (ROX). FIG. 8 is a chart showing the correlation between the relative fluorescent units and the template concentration in an example of the present disclosure. As shown in FIG. 8, a linear correlation between the relative fluorescent units and the template concentration was found.

### Example III. Fragments without the Correct Sequences are not Detected

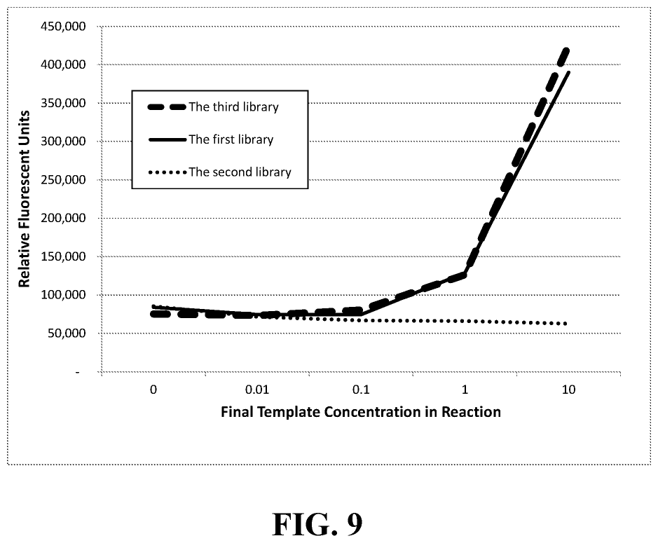

Three libraries were tested. The first library contained target nucleic acid fragments (SEQ ID NO: 32) containing SEQ ID NO. 1 on its 5′ end and SEQ ID NO. 4 on its 3′ end. The second library contained complementary strands (SEQ ID NO: 35) of the target nucleic acid fragment in the first library. The third library contained both the target nucleic acid fragments and the complementary strands. To each of the three libraries, DNA polymerase was added in combination with an extension primer (SEQ ID NO. 20) and a detectably-labeled probe (SEQ ID NO. 13). Incubation was performed at 95 degrees for 60 seconds, 60 degrees for 60 seconds, followed by reading of fluorescence. Incubation and reading was performed on a QuantStudio 6 Pro (ThermoFisher, Catalog no. A43180) and plotted as Relative Fluorescent units. FIG. 9 is a chart showing the correlation between the relative fluorescent units and the final template concentration in reaction in an example of the present disclosure. The complementary strands did not contain binding sites for the extension primer and the detectably-labeled probe. As shown in FIG. 9, the complementary strands without the binding sites for the extension primer and the detectably-labeled probe were not detected.

### Example IV. Correlation of NGS Sequencing Reads to Calculated Concentration

Eighteen different libraries created using Nextera XT (Illumina, Catalog no FC-131-1096). Equal volumes of each library were pooled and sequenced on a NextSeq 550 (Illumina, Catalog no SY-415-1002).

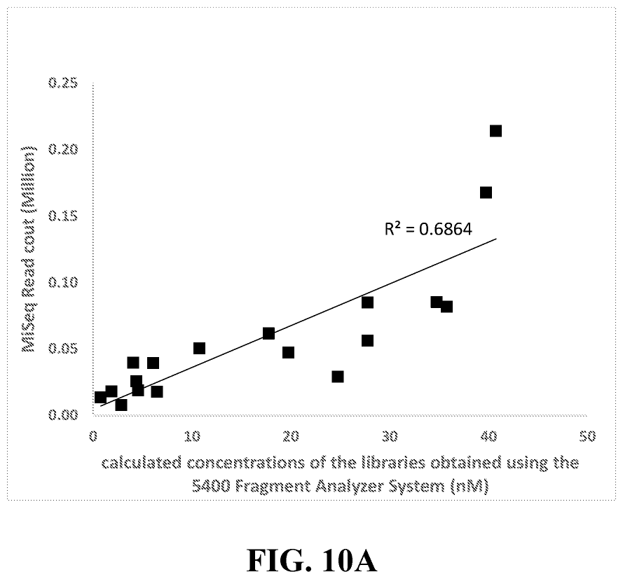

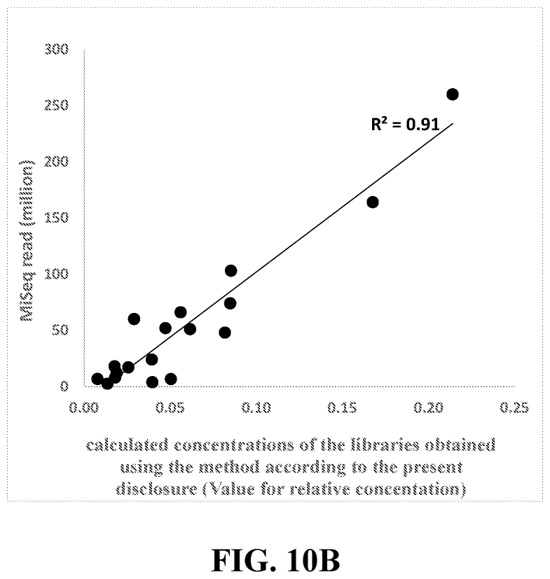

Individual libraries were quantitated on a 5400 Fragment Analyzer System (Agilent, Catalog no M5312AA) and average fragment size, mass, and molarity were calculated (FIG. 10A). FIG. 10A is chart showing the correlation between the MISeq read number and calculated concentrations of the libraries obtained using the 5400 Fragment Analyzer System.

Individual libraries were quantitated using a method according to the present disclosure (FIG. 10B). DNA polymerase was added in combination with an extension primer (SEQ ID NO: 20) and a detectably-labeled probe (SEQ ID NO: 13). Incubation was performed at 95 degrees for 60 seconds, 60 degrees for 60 seconds, followed by reading of fluorescence. Incubation and reading was performed on a 7900HT Fast Real-Time PCR System (ThermoFisher, Catalog no. 4329001). FIG. 10B is chart showing the correlation between the MISeq read number and calculated concentrations of the libraries obtained using the method according to the present disclosure. As shown in FIG. 10A and FIG. 10B, a significantly improved correlation between the read number and the calculated concentrations is obtain in the method according to the present disclosure, as compared to the 5400 Fragment Analyzer method.

### Example V. Workflow Time

Table 1 shows a comparison between the workflow time using a method according to the present disclosure and the workflow time using various other methods. As shown in Table 1, a significantly shorten workflow time was achieved in the method according to the present disclosure.

Workflow times were taken from supplier recommendations or estimations based on protocols and times are listed for 11 samples using a single instrument. KAPA qPCR Library Quantification Kit (Roche, Catalog no. KK4824). Qubit 4 Fluorometer (ThermoFisher, Catalog no. Q33226). 5400 Fragment Analyzer System (Agilent, Catalog no M5312AA). 2100 Bioanalyzer Instrument (Agilent, Catalog no. 3428300). QuantStudio 6 Pro (ThermoFisher, Catalog no. A43180).

TABLE 1
Calculated workflow times based on supplier recommendations

| A method | | | | | | | |
|---|---|---|---|---|---|---|---|
| according to | | KAPA qPCR | | | | | |
| the present | | Library | | 5400 Fragment | | | |
| disclosure | | Quantification Kit | | Analyzer System | | 2100 Bioanalyzer | |
| Prepare | 10 min | Prepare | 5 min | Estimate | 15 min | Estimate | 15 min |
| samples in | | qPCR/Primer | | concentration | | concentration | |
| plate | | Mix | | (mass) of each | | (mass) of each | |
| | | | | library using a | | library using a | |
| | | | | Fluorometer | | Fluorometer | |
| Run protocol | 3 min | Make 1:1,000 | 10 min | Prepare | 30 min | Equilibrate Dye | 30 min |
| | | and 1:10,000 | | samples | | and gel matrix | |
| | | fold dilutions | | | | to room | |
| | | of DNA | | | | temperature | |
| | | libraries | | | | | |
| Analyze | 5 min | Prepare qPCR | 10 min | Run instrument | 90 min | Prepare gel | 11 min |
| Data and | | plate | | | | matrix and | |
| Calculate | | | | | | centrifuge and | |
| library | | | | | | prime chip | |
| concentration | | | | | | | |
| — | — | Run qPCR | 90 min | Analyze Data | 30 min | | |
| | | protocol | | | | | |
| — | — | Analyze Data | 20 min | Calculate | 10 min | Load markers | 8 min |
| | | | | library | | and samples, | |
| | | | | concentration | | mix and | |
| | | | | | | incubate | |
| — | — | Fragment | 139 min | — | — | Run instrument | 45 min |
| | | sizing using | | | | | |
| | | 2100 | | | | | |
| | | Bioanalyzer | | | | | |
| — | — | Calculate | 10 min | — | — | Analyze Data | 20 min |
| | | library | | | | | |
| | | concentration | | | | | |
| — | — | — | — | — | — | Calculate | 10 min |
| | | | | | | library | |
| | | | | | | concentration | |
| Total Time | 18 min | Total Time | 284 min | Total time | 175 Min | Total time | 139 min |
| | | | (4.7 hrs) | | (2.9 hrs) | | (2.3 hrs) |

### Example VI. Workflow Time

Table 2 shows a comparison between the workflow time using a method according to the present disclosure and the workflow time using various other methods. As shown in Table 1, a significantly shorten workflow time was achieved in the method according to the present disclosure.

Workflow times were taken from supplier recommendations or estimations based on protocols and times are listed for 48 samples using a single instrument. KAPA qPCR Library Quantification Kit (Roche, Catalog no. KK4824). Qubit 4 Fluorometer (ThermoFisher, Catalog no. Q33226). 5400 Fragment Analyzer System (Agilent, Catalog no M5312AA). 2100 Bioanalyzer Instrument (Agilent, Catalog no. 3428300). QuantStudio 6 Pro (ThermoFisher, Catalog no. A43180).

TABLE 2
Calculated workflow times based on supplier recommendations

| A method | | | | | | | |
|---|---|---|---|---|---|---|---|
| according to the | | | | | | | |
| present | | KAPA qPCR Library | | 5400 Fragment | | | |
| disclosure | | Quantification Kit | | Analyzer System | | 2100 Bioanalyzer | |
| Prepare | 20 min | Prepare | 5 min | Estimate | 45 min | Estimate | 45 min |
| samples in | | qPCR/Primer | | concentration | | concentration | |
| plate | | Mix | | (mass) of | | (mass) of each | |
| | | | | each library | | library using a | |
| | | | | using a | | Fluorometer | |
| | | | | Fluorometer | | | |
| Run | 6 min | Make 1:1000 | 20 min | Prepare | 45 min | Equilibrate | 30 min |
| protocol (2 | | dilution of ds | | samples | | Dye and gel | |
| runs of 3 | | DNA library | | | | matrix to room | |
| minutes | | | | | | temperature | |
| each) | | | | | | | |
| Analyze | 5 min | Prepare | 20 min | — | — | Prepare gel | 11 min |
| Data and | | qPCR plates | | | | matrix and | |
| Calculate | | (only 16 | | | | centrifuge and | |
| library | | samples per | | | | prime chip | |
| concentration | | 96 well | | | | | |
| | | plate) | | | | | |
| — | — | Run qPCR | 270 min | — | — | Load markers | 8 min |
| | | protocol (90 | | | | and samples, | |
| | | min each; | | | | mix and | |
| | | three times) | | | | incubate | |
| — | — | Analyze Data | 20 min | Run | 90 min | Run instrument | 225 min |
| | | | | instrument | | (45 min each; | |
| | | | | | | instrument run | |
| | | | | | | 5 times) | |
| — | — | Fragment | 265 min | Analyze Data | 75 min | | |
| | | sizing using | | | | | |
| | | 5400 | | | | | |
| | | Fragment | | | | | |
| | | Analyzer | | | | | |
| | | System | | | | | |
| — | — | Calculate | 10 min | Calculate | 10 min | Analyze Data | 20 min |
| | | library | | library | | | |
| | | concentration | | concentration | | | |
| — | — | — | — | — | — | Calculate | 10 min |
| | | | | | | library | |
| | | | | | | concentration | |
| Total Time | 31 min | Total Time | 610 min | Total time | 265 min | Total time | 349 min |
| | | | (10.2 hrs) | | (4.4 hrs) | | (5.8 hrs) |

### Example VII. Detection of Fluorescence can be Performed Using Fluorometers

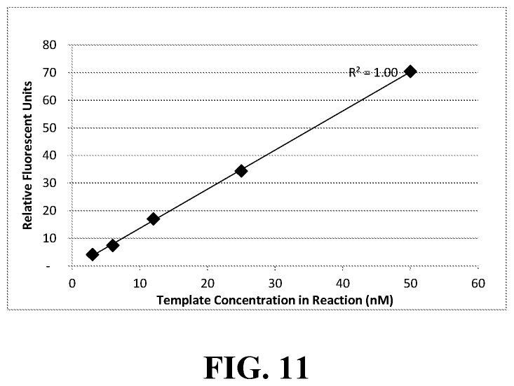

A library having oligonucleotides (SEQ ID NO: 33) containing SEQ ID NO. 1 on its 5′ end and SEQ ID NO. 4 on its 3′ end was serially diluted and put into reactions to get final concentrations from 0.08 nM to 2.5 nM as shown on the X-axis in FIG. 11. DNA polymerase was added in combination with an extension primer (SEQ ID NO: 20) and a detectably-labeled probe (SEQ ID NO: 12). Incubation was performed at 95 degrees for 60 seconds, 60 degrees for 60 seconds using a Veriti 96-Well Thermal Cycler (ThermoFisher, Catalog no. 4375786) followed by reading of fluorescence using a Qubit 4 Fluorometer (ThermoFisher, Catalog no. Q33226). FIG. 11 is a chart showing the correlation between the relative fluorescent units detected using a Qubit 4 Fluorometer and the template concentration in an example of the present disclosure. As shown in FIG. 11, a linear correlation between the relative fluorescent units and the template concentration was found.

### Example VIII. Determination of an Average Size of Target Nucleic Acid Fragments

Five different libraries which contained flow cell binding sequences (e.g., SEQ ID NO:1 on 5′ end and SEQ ID NO: 4 on 3′ end) were added to reactions at a final concentration of 20 nM. DNA polymerase was added in combination with an extension oligonucleotide, a probe labelled with Cy3, and SYBR Gold intercalating dye. Incubation was performed at 95 degrees for 60 seconds, 60 degrees for 60 seconds, repeated 5 times and followed by reading of fluorescence. Incubation and reading was performed on a QuantStudio 3 (ThermoFisher, Catalog no. A28137) and plotted as Relative Fluorescent units after normalization with a passive reference dye 6-carboxy-X-rhodamine (ROX).

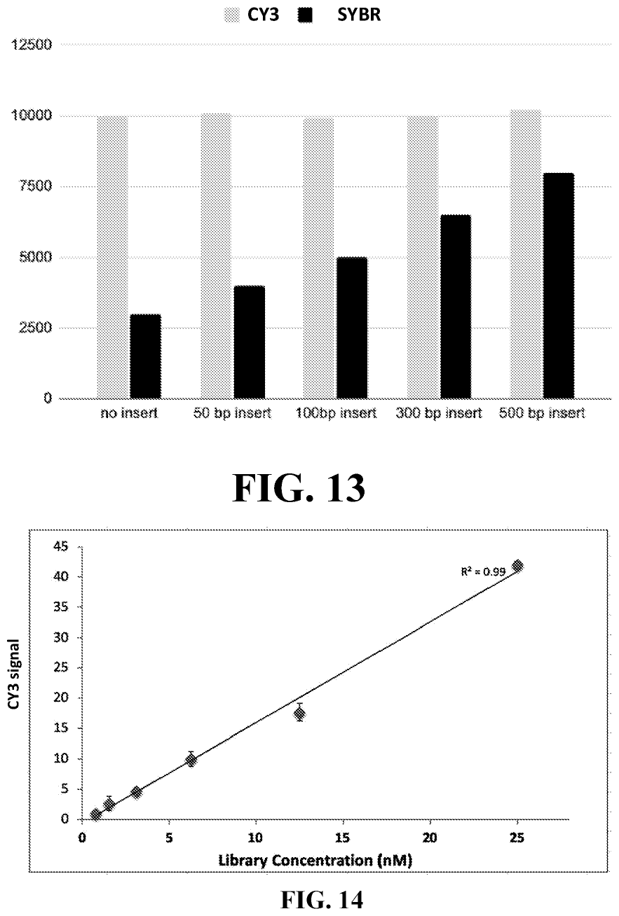

Fluorescence values are respectively obtained at SYBR channel, indicating the mass of the plurality of target nucleic acid fragments (mass); and at CY3 channel, indicating the number of the plurality of target nucleic acid fragments (molarity). FIG. 13 illustrates fluorescence values for the plurality of target nucleic acid fragments with inserts of different lengths. As shown in FIG. 13, fluorescence values obtained at SYBR channel increase as the lengths of inserts increase, the fluorescence values obtained at SYBR channel are positively correlated with the mass of the plurality of target nucleic acid fragments.

A correlation factor may be obtained using Equation (4):

$$Correlation factor = \frac{SYBR fluorescence value}{average size \times molarity} ; \qquad ( 4 )$$

wherein the average size may be expressed as a number of base pairs; the molarity may be expressed as concentrations (e.g., in nM).

FIG. 14 illustrates a correlation between fluorescence values obtained at CY3 channel and nucleic acid molarities in the libraries. As shown in FIG. 14, the slope and the intercept were obtained from a curve obtained using known reference libraries. The fluorescence values of the five reference libraries are plotted to obtain a curve, with a slope of 0.017, an intercept of −10.058, and an R2 value of 1.000. The molarities of the unknown libraries were calculated using Equation (5):

Molarity=(CY3 fluorescence value×slope)+intercept (5).

Once the correlation factor is calibrated using known reference libraries (with known average sizes and known molarities), an average size of a library (e.g., an unknown library) can be calculated using Equation (6):

Table 1 shows fluorescence values obtained for five reference libraries.

$$Average size = \frac{SYBR fluorescence value}{correlation factor \times molarity} ; \qquad ( 6 )$$

| | Expected | Insert | fluorescence | fluorescence |
|---|---|---|---|---|
| | concen- | sizes | values obtained | values |
| Reference | trations | (base | at SYBR | obtained at |
| Libraries | (nM) | pairs) | channel | CY3 channel |
| 1 | 2 | 500 | 1950 | 725 |
| 2 | 5 | 400 | 4200 | 900 |
| 3 | 10 | 300 | 6150 | 1200 |
| 4 | 20 | 200 | 7934 | 1800 |
| 5 | 30 | 100 | 6177 | 2400 |

Table 2 summarizes the correlation factors respectively for the five reference libraries.

| Reference Libraries | Correlation factor |
|---|---|
| 1 | 1.95 |
| 2 | 2.10 |
| 3 | 2.05 |
| 4 | 1.98 |
| 5 | 2.06 |
| Average | 2.03 |

The fluorescence values obtained using reference libraries were then used for determining molarities and average sizes of unknown libraries. Table 3 summarizes fluorescence values of the four unknown libraries.

| Unknown | Fluorescence values at | Fluorescence values |
|---|---|---|
| libraries | SYBR channel | at CY3 channel |
| A | 4255 | 1534 |
| B | 21565 | 2011 |
| C | 4877 | 923 |
| D | 4988 | 1255 |

The average sizes of the unknown libraries were calculated using Equation (6).

Table 4 summarizes the molarities and average sizes of the unknown libraries determined according to the methods of the present disclosure.

| Unknown | | Average |
|---|---|---|
| libraries | Molarities | sizes |
| A | 15.55 | 135 |
| B | 23.52 | 452 |
| C | 5.35 | 449 |
| D | 10.90 | 226 |

### Example IX. Determination of an Average Size of Target Nucleic Acid Fragments

A single library (“control reference fragments”) which contained flow cell binding sequences (e.g., SEQ ID NO:1 on 5′ end and SEQ ID NO: 4 on 3′ end) was added to reactions at a final concentration of 5, 10, 20 and 30 nM. DNA polymerase was added in combination with an extension oligonucleotide, a probe labelled with VIC, and SYBR Gold intercalating dye. Incubation was performed at 95 degrees for 60 seconds, 60 degrees for 60 seconds, repeated 5 times and followed by reading of fluorescence. Incubation and reading was performed on a QuantStudio 3 (ThermoFisher, Catalog no. A28137) and plotted as Relative Fluorescent units after normalization with a passive reference dye 6-carboxy-X-rhodamine (ROX).

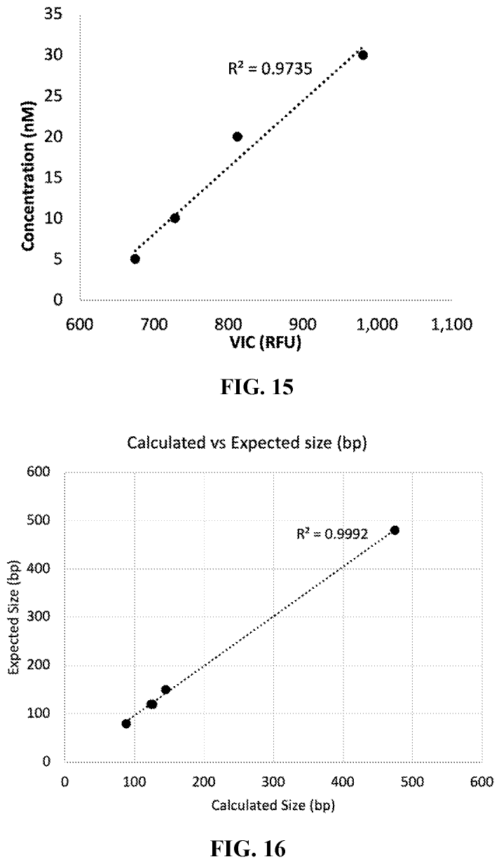

Fluorescence values are respectively obtained at SYBR channel, indicating the mass of the plurality of target nucleic acid fragments (mass); and at VIC channel, indicating the number of the plurality of target nucleic acid fragments (molarity). FIG. 15 illustrates fluorescence values for the plurality of target nucleic acid fragments at different concentrations. As shown in FIG. 15, relative fluorescence values (RFU) obtained at VIC channel increase as the concentrations increase, the fluorescence values obtained at SYBR channel are positively correlated with the mass of the plurality of target nucleic acid fragments.

A correlation factor may be obtained using Equation (4):

$$Correlation factor = \frac{SYBR fluorescence value}{average size \times molarity} ; \qquad ( 4 )$$

wherein the average size may be expressed as a number of base pairs; the molarity may be expressed as concentrations (e.g., in nM).

Five different libraries which contained flow cell binding sequences (e.g., SEQ ID NO:1 on 5′ end and SEQ ID NO: 4 on 3′ end) were added to reactions at a final concentration of approximately 20 nM. DNA polymerase was added in combination with an extension oligonucleotide, a probe labelled with VIC, and SYBR Gold intercalating dye. Incubation was performed at 95 degrees for 60 seconds, 60 degrees for 60 seconds, repeated 5 times and followed by reading of fluorescence. Incubation and reading was performed on a QuantStudio 3 (ThermoFisher, Catalog no. A28137) and plotted as Relative Fluorescent units after normalization with a passive reference dye 6-carboxy-X-rhodamine (ROX).

Fluorescence values are respectively obtained at SYBR channel, indicating the mass of the plurality of target nucleic acid fragments (mass); and at VIC channel, indicating the number of the plurality of target nucleic acid fragments (molarity). Table 5 illustrates fluorescence values for the plurality of target nucleic acid fragments with inserts of different lengths. As shown in Table 5, fluorescence values obtained at SYBR channel increase as the lengths of inserts increase,

TABLE 5
Fluorescence values for the plurality of target nucleic
acid fragments with inserts of different lengths.

| Unknown | | |
|---|---|---|
| libraries | VIC RFU | SYBR RFU |
| A | 862 | 3,305 |
| B | 871 | 4,889 |
| C | 800 | 3,563 |
| D | 833 | 4,847 |
| E | 780 | 12,318 |

As shown in FIG. 15, the slope and the intercept were obtained from a curve obtained using control reference fragments. The fluorescence values of the five reference libraries are plotted to obtain a curve, with a slope of 0.082, an intercept of −48.947, and an R2 value of 0.975. The molarities of the unknown libraries were calculated using Equation (7):

Molarity=(VIC fluorescence value×slope)+intercept (7).

Once the correlation factor is calibrated using known control reference fragments (with known average sizes and known molarities), an average size of a library (e.g., an unknown library) can be calculated using Equation (6):

$$Average size = \frac{SYBR fluorescence value}{correlation factor \times molarity} ; \qquad ( 6 )$$

Table 6 summarizes the molarities and average sizes of the unknown libraries determined according to the methods of the present disclosure.

| Unknown | | |
|---|---|---|
| libraries | Molarities | Average sizes |
| A | 21.7 | 88 |
| B | 22.5 | 126 |
| C | 16.7 | 124 |
| D | 19.4 | 145 |
| E | 15.0 | 474 |

FIG. 16 Illustrates the correlation between known fragment sizes and calculated fragment sizes.

The foregoing description of the embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

What is claimed is:

1. A method for quantitating a plurality of nucleic acid molecules comprising a plurality of target nucleic acid fragments, the method comprising:
contacting a plurality of detectably-labeled probes and a plurality of extension primers with the plurality of nucleic acid molecules, wherein each of the plurality of detectably-labeled probes comprises a first labeled nucleic acid domain comprising a first label;
producing an extension product of each of the plurality of target nucleic acid fragments by extending a respective one of the plurality of extension primers with a polymerase;
hydrolyzing each of the plurality of detectably-labeled probes hybridized to the respective one of the target nucleic acid fragments during extending the respective one of the plurality of extension primers with the polymerase;
detecting a first signal produced as a result of hydrolyzing the plurality of detectably-labeled probes; and
calculating a number of the plurality of target nucleic acid fragments based on signals detected upon extension reactions.

2. The method of claim 1, wherein calculating the number of the plurality of target nucleic acid fragments is based on the first signal detected upon a single cycle of extension reactions.

3. The method of claim 1, wherein a total number of hydrolyzed detectably-labeled probes is substantially same as a total number of the plurality of target nucleic acid fragments prior to contacting the plurality of detectably-labeled probes and the plurality of extension primers with the plurality of nucleic acid molecules.

4. The method of claim 1, wherein, subsequent to initially contacting the plurality of detectably-labeled probes and the plurality of extension primers with the plurality of nucleic acid molecules, and prior to detecting the first signal, no additional cycle of extension reactions is performed other than a single cycle of extension reactions.

5. The method of claim 1, wherein each of the plurality of target nucleic acid fragments comprises a first adapter sequence, a target insert sequence, and a second adapter sequence;
the first adapter sequence is linked to the second adapter sequence through the target insert sequence;
hydrolyzing each of the plurality of detectably-labeled probes comprises hydrolyzing a detectably-labeled probe hybridized to at least a portion of the first adapter sequence; and
extending the respective one of the plurality of extension primers comprises extending an extension primer hybridized to at least a portion of the second adapter sequence.

6. The method of claim 1, wherein the plurality of nucleic acid molecules further comprise a plurality of adapter molecules; and
each of the plurality of adapter molecules comprises a first adapter sequence directly linked to a second adapter sequence;
wherein the method further comprises contacting a plurality of hybridizing oligonucleotides with the plurality of nucleic acid molecules;
wherein each of the plurality of hybridizing oligonucleotides comprises a sequence complementary to a contiguous domain of a respective one of the plurality of adapter molecules.

7. The method of claim 6, wherein each of the plurality of hybridizing oligonucleotides comprises a blocker nucleic acid domain;
subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments; and
hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain.

8. The method of claim 7, wherein the contiguous domain comprises a portion of the first adapter sequence and a portion of the second adapter sequence directly adjacent to each other.

9. The method of any one of claims 1 to 8, wherein the first signal is a first fluorescent signal, and the signals detected upon the extension reactions are fluorescent signals comprising the first fluorescent signal.

10. The method of any one of claims 1 to 9, wherein each of the plurality of detectably-labeled probes further comprises a second labeled nucleic acid domain comprising a second label;
the first labeled nucleic acid domain is a first reporter domain;
the second labeled nucleic acid domain is a first quencher domain; and
the plurality of detectably-labeled probes are hydrolyzed to release at least one of the first label or the second label.

11. The method of any one of claims 1 to 9, wherein each of the plurality of detectably-labeled probes further comprises a second labeled nucleic acid domain comprising a second label;
the first label and the second label are two spectrally similar or identical reporters; and
the plurality of detectably-labeled probes are hydrolyzed to release at least one of the first label or the second label.

12. The method of any one of claims 1 to 9, wherein the first labeled nucleic acid domain is a first reporter domain;
each of the plurality of detectably-labeled probes comprises a quenching nucleotide that quenches an energy from the first label in an excited state; and
the plurality of detectably-labeled probes are hydrolyzed to release at least one of the quenching nucleotide or the first label.

13. The method of any one of claims 7 to 8, wherein each of the plurality of hybridizing oligonucleotides further comprises a third labeled nucleic acid domain comprising a third label;
subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments;
hydrolyzation of each of the plurality of hybridizing oligonucleotides hybridized to the contiguous domain and hydrolyzation of each of the plurality of detectably-labeled probes hybridized to the first adapter sequence of each of the plurality of adapter molecules are blocked by a blocking moiety in the blocker nucleic acid domain; and
the plurality of hybridizing oligonucleotides are partially hydrolyzed;
wherein the method further comprises detecting a second signal produced as a result of partially hydrolyzing the plurality of hybridizing oligonucleotides; and
the first signal and the second signal are distinguishably detected.

14. The method of claim 13, wherein each of the plurality of hybridizing oligonucleotides further comprises a fourth labeled nucleic acid domain comprising a fourth label;
the third labeled nucleic acid domain is a second reporter domain;
the fourth labeled nucleic acid domain is a second quencher domain;
the second reporter domain and the second quencher domain are linked by the blocker nucleic acid domain; and
the plurality of hybridizing oligonucleotides are partially hydrolyzed to release at least one of the third label or the fourth label.

15. The method of claim 13, wherein each of the plurality of hybridizing oligonucleotides further comprises a fourth labeled nucleic acid domain comprising a fourth label;
the third label and the fourth label are two spectrally similar or identical reporters; and
the plurality of hybridizing oligonucleotides are partially hydrolyzed to release at least one of the third label or the fourth label.

16. The method of claim 13, wherein the third labeled nucleic acid domain is a third reporter domain;
each of the plurality of hybridizing oligonucleotides comprises a quenching nucleotide that quenches an energy from the third label in an excited state; and
the plurality of hybridizing oligonucleotides are partially hydrolyzed to release at least one of the quenching nucleotide or the third label.

17. The method of claim 13, wherein the first signal is a first fluorescent signal, the second signal is a second fluorescent signal, and the signals detected upon the extension reactions are fluorescent signals comprising the first fluorescent signal and the second fluorescent signal.

18. The method of claim 6, wherein each of the plurality of hybridizing oligonucleotides comprises a third labeled nucleic acid domain comprising a third label; and
subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments;
wherein the method further comprises:
producing an extension product of each of the plurality of adapter molecules by extending a respective one of the plurality of extension primers in the extension reactions with the polymerase;
hydrolyzing each of the plurality of hybridizing oligonucleotides hybridized to the contiguous domain and hydrolyzing each of the plurality of detectably-labeled probes hybridized to the respective one of the adapter molecules, during producing an extension product of each of the plurality of adapter molecules;
detecting a second signal produced as a result of hydrolyzing the plurality of hybridizing oligonucleotides; and
the first signal and the second signal are distinguishably detected.

19. The method of claim 18, wherein each of the plurality of hybridizing oligonucleotides further comprises a fourth labeled nucleic acid domain comprising a fourth label;
the third labeled nucleic acid domain is a second reporter domain;
the fourth labeled nucleic acid domain is a second quencher domain; and
the plurality of hybridizing oligonucleotides are hydrolyzed to release at least one of the third label or the fourth label.

20. The method of claim 18, wherein each of the plurality of hybridizing oligonucleotides further comprises a fourth labeled nucleic acid domain comprising a fourth label;
the third label and the fourth label are two spectrally similar or identical reporters; and
the plurality of hybridizing oligonucleotides are hydrolyzed to release at least one of the third label or the fourth label.

21. The method of claim 18, wherein the third labeled nucleic acid domain is a third reporter domain;
each of the plurality of hybridizing oligonucleotides comprises a quenching nucleotide that quenches an energy from the third label in an excited state; and
the plurality of hybridizing oligonucleotides are hydrolyzed to release at least one of the quenching nucleotide or the third label.

22. The method of claim 18, wherein the first signal is a first fluorescent signal, the second signal is a second fluorescent signal, and the signals detected upon the extension reactions are fluorescent signals comprising the first fluorescent signal and the second fluorescent signal.

23. The method of any one of claims 5 to 8, and 13-22, wherein the first adapter sequence comprises a first flow cell binding sequence;
the second adapter sequence comprises a second flow cell binding sequence;
wherein, hydrolyzing each of the plurality of detectably-labeled probes comprises hydrolyzing a detectably-labeled probe hybridized to at least a portion of the first flow cell binding sequence; and
extending the respective one of the plurality of extension primers comprises extending an extension primer hybridized to at least a portion of the second flow cell binding sequence.

24. The method of claim 8, wherein the first adapter sequence comprises a first sequencing primer binding sequence;
the second adapter sequence further comprises a second sequencing primer binding sequence;
the first sequencing primer binding sequence is directly linked to the second sequencing primer binding sequence; and
the contiguous domain comprises a portion of the first sequencing primer binding sequence and a portion of the second sequencing primer binding sequence directly adjacent to each other.

25. The method of any one of claims 1 to 24, the method further comprising preparing for solid phase attachment by diluting the plurality of target nucleic acid fragments to a predetermined concentration.

26. The method of any one of claims 1 to 25, wherein the plurality of target nucleic acid fragments are a plurality of target double-strand DNA fragments;
wherein, prior to producing the extension product of each of the plurality of target nucleic acid fragments, the method further comprises denaturing the plurality of target double-strand DNA fragments.

27. A method for quantitating a plurality of nucleic acid molecules comprising a plurality of target nucleic acid fragments, the method comprising:
contacting a plurality of extension primers and a plurality of nucleic acid binding dye molecules with the plurality of nucleic acid molecules;
producing an extension product of each of the plurality of target nucleic acid fragments by extending a respective one of the plurality of extension primers with a polymerase;
subsequent to producing the extension product of each of the plurality of target nucleic acid fragments, measuring a signal produced by the plurality of nucleic acid binding dye molecules; and
estimating an average size of the plurality of target nucleic acid fragments based on a number of the plurality of target nucleic acid fragments and the signal produced by the plurality of nucleic acid binding dye molecules.

28. The method of claim 27, further comprising:
contacting a plurality of detectably-labeled probes and the plurality of extension primers with the plurality of nucleic acid molecules, wherein each of the plurality of detectably-labeled probes comprises a first labeled nucleic acid domain comprising a first label;
producing the extension product of each of the plurality of target nucleic acid fragments by extending the respective one of the plurality of extension primers with a polymerase;
hydrolyzing each of the plurality of detectably-labeled probes hybridized to the respective one of the target nucleic acid fragments during extending the respective one of the plurality of extension primers with the polymerase;
detecting a first signal produced as a result of hydrolyzing the plurality of detectably-labeled probes; and
calculating the number of the plurality of target nucleic acid fragments based on signals detected upon extension reactions.

29. The method of claim 27 or claim 28, wherein estimating an average size of the plurality of target nucleic acid fragments comprises:
determining a correlation factor using signals respectively produced by a plurality of reference nucleic acid libraries;
estimating the average size of the plurality of target nucleic acid fragments according to the following Equation:
$$Average size = \frac{S}{correlation factor \times N} ;$$
wherein S stands for the signal produced by the plurality of nucleic acid binding dye molecules, and N stands for the number of the plurality of target nucleic acid fragments.

30. The method of any one of claims 27 to 29, wherein the plurality of nucleic acid binding dye molecules are a plurality of double-stranded nucleic acid intercalating dye molecules.

31. The method of any one of claims 27 to 29, wherein the plurality of nucleic acid binding dye molecules are a plurality of single-stranded nucleic acid dye molecules.

32. The method of any one of claims 27 to 29, wherein a respective one of the plurality of nucleic acid binding dye molecules is a fluorescence dye selected from the group consisting of ethidium bromide, SYBR Green, SYBR Gold, SYBR Safe, GelRed, GelGreen, and Diamond™ Nucleic Acid Dye.

33. A method of sequencing a nucleic acid sample, comprising:
generating a plurality of nucleic acid molecules comprising a plurality of target nucleic acid fragments using a nucleic acid sample;
contacting a plurality of detectably-labeled probes and a plurality of extension primers with the plurality of nucleic acid molecules, wherein each of the plurality of detectably-labeled probes comprises a first labeled nucleic acid domain comprising a first label;
producing an extension product of each of the plurality of target nucleic acid fragments by extending a respective one of the plurality of extension primers with a polymerase;
hydrolyzing each of the plurality of detectably-labeled probes hybridized to the respective one of the target nucleic acid fragments during extending the respective one of the plurality of extension primers with the polymerase;
detecting a first signal produced as a result of hydrolyzing the plurality of detectably-labeled probes;
calculating a number of the plurality of target nucleic acid fragments based on signals detected upon a single cycle of extension reactions;
diluting the plurality of target nucleic acid fragments to a predetermined concentration; and
sequencing at least one portion of the plurality of target nucleic acid fragments.

34. A method for quantitating a plurality of nucleic acid molecules comprising a plurality of target nucleic acid fragments and a plurality of adapter molecules, the method comprising:
contacting a plurality of detectably-labeled probes, a plurality of extension primers, and a plurality of hybridizing oligonucleotides with the plurality of nucleic acid molecules, wherein each of the a plurality of detectably-labeled probes comprises a first labeled nucleic acid domain comprising a first label, and each of the a plurality of hybridizing oligonucleotides comprises a blocker nucleic acid domain;
producing an extension product of each of the plurality of target nucleic acid fragments by extending a respective one of the plurality of extension primers with a polymerase;
hydrolyzing each of the plurality of detectably-labeled probes hybridized to the respective one of the target nucleic acid fragments during extending the respective one of the plurality of extension primers with the polymerase; and
detecting a first signal produced as a result of hydrolyzing the plurality of detectably-labeled probes.

35. The method of claim 34, wherein each of the plurality of hybridizing oligonucleotides comprises a sequence complementary to a contiguous domain of a respective one of the plurality of adapter molecules;
subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments;
each of the plurality of target nucleic acid fragments comprises a first adapter sequence, a target insert sequence, and a second adapter sequence, the first adapter sequence is linked to the second adapter sequence through the target insert sequence;
each of the plurality of adapter molecules comprises a first adapter sequence directly linked to a second adapter sequence;
hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain.

36. The method of claim 35, wherein the contiguous domain comprises a portion of the first adapter sequence and a portion of the second adapter sequence directly adjacent to each other.

37. The method of any one of claims 35 to 36, wherein each of the plurality of hybridizing oligonucleotides further comprises a third labeled nucleic acid domain comprising a third label;
subsequent to contacting the plurality of hybridizing oligonucleotides with the plurality of nucleic acid molecules, each of the plurality of hybridizing oligonucleotides hybridizes to the contiguous domain of the respective one of the plurality of adapter molecules, but not to the plurality of target nucleic acid fragments;
hydrolyzation of each of the plurality of hybridizing oligonucleotides hybridized to the contiguous domain and hydrolyzation of each of the plurality of detectably-labeled probes hybridized to the first adapter sequence of a respective one of the plurality of adapter molecules are blocked by a blocking moiety in the blocker nucleic acid domain; and
the plurality of hybridizing oligonucleotides are partially hydrolyzed;
wherein the method further comprises detecting a second signal produced as a result of partially hydrolyzing the plurality of hybridizing oligonucleotides; and
the first signal and the second signal are distinguishably detected.

38. The method of claim 37, wherein the first signal is a first fluorescent signal, the second signal is a second fluorescent signal, and the signals detected upon the single cycle of extension reactions are fluorescent signals comprising the first fluorescent signal and the second fluorescent signal.

39. The method of claim 37, wherein each of the plurality of hybridizing oligonucleotides further comprises a fourth labeled nucleic acid domain comprising a fourth label;
the third labeled nucleic acid domain is a second reporter domain;
the fourth labeled nucleic acid domain is a second quencher domain;
the second reporter domain and the second quencher domain are linked by the blocker nucleic acid domain; and
the plurality of hybridizing oligonucleotides are partially hydrolyzed to release at least one of the third label or the fourth label.

40. The method of claim 37, wherein each of the plurality of hybridizing oligonucleotides further comprises a fourth labeled nucleic acid domain comprising a fourth label;
the third label and the fourth label are two spectrally similar or identical reporters; and
the plurality of hybridizing oligonucleotides are partially hydrolyzed to release at least one of the third label or the fourth label.

41. The method of claim 37, wherein the third labeled nucleic acid domain is a third reporter domain;
each of the plurality of hybridizing oligonucleotides comprises a quenching nucleotide that quenches an energy from the third label in an excited state; and
the plurality of hybridizing oligonucleotides are partially hydrolyzed to release at least one of the quenching nucleotide or the third label.

42. A mixture, comprising:
a plurality of nucleic acid molecules comprising a plurality of target nucleic acid fragments;
a plurality of detectably-labeled probes; and
a plurality of hybridized adapter molecules;
wherein each of the plurality of detectably-labeled probes comprises a first labeled nucleic acid domain comprising a first label;
each of the plurality of target nucleic acid fragments comprises a first adapter sequence, a target insert sequence, and a second adapter sequence;
the first adapter sequence is linked to the second adapter sequence through the target insert sequence;
at least a portion of each of the plurality of detectably-labeled probes is complementary to at least a portion of the first adapter sequence;
wherein each of the plurality of hybridized adapter molecules comprises:
the first adapter sequence;
the second adapter sequence; and
a partially hydrolyzed hybridizing oligonucleotide, at least a portion of which is hybridized to a domain of a respective one of a plurality of adapter molecules;
wherein the first adapter sequence is directly linked to the second adapter sequence; and
the domain comprises a portion of the first adapter sequence and a portion of the second adapter sequence directly adjacent to each other.

43. The mixture of claim 42, wherein each of the plurality of hybridized adapter molecules further comprises:
one of the plurality of detectably-labeled probes, at least a portion of which is hybridized to at least a portion of the first adapter sequence; and
one of a plurality of extension primers, at least a portion of which is hybridized to at least a portion of the at least a portion of the second adapter sequence.

44. The mixture of claim 42, wherein each of the plurality of detectably-labeled probes further comprises a second labeled nucleic acid domain comprising a second label;
the first labeled nucleic acid domain is a first reporter domain; and
the second labeled nucleic acid domain is a first quencher domain.

45. The mixture of claim 42, wherein each of the plurality of detectably-labeled probes further comprises a second labeled nucleic acid domain comprising a second label;
the first label and the second label are two spectrally similar or identical reporters.

46. The mixture of claim 42, wherein the first labeled nucleic acid domain is a first reporter domain; and
each of the plurality of detectably-labeled probes comprises a quenching nucleotide that quenches an energy from the first label in an excited state.

47. The mixture of claim 42, wherein the first adapter sequence comprises a first flow cell binding sequence;
at least a portion of each of the plurality of detectably-labeled probes is complementary to at least a portion of the first flow cell binding sequence.

48. The mixture of claim 42, further comprising a plurality of extension primers;
wherein at least a portion of each of the plurality of extension primers is complementary to at least a portion of the second adapter sequence.

49. The mixture of claim 48, wherein the second adapter sequence comprises a second flow cell binding sequence; and
at least a portion of each of the plurality of extension primers is complementary to at least a portion of the second flow cell binding sequence.

50. The mixture of any one of claims 42 to 49, further comprising a plurality of hybridizing oligonucleotides;
wherein each of the plurality of hybridizing oligonucleotides comprises a blocker nucleic acid domain.

51. The mixture of claim 50, wherein each of the plurality of hybridizing oligonucleotides further comprises a third labeled nucleic acid domain comprising a third label.

52. The mixture of claim 51, wherein each of the plurality of hybridizing oligonucleotides further comprises a fourth labeled nucleic acid domain comprising a fourth label;
the third labeled nucleic acid domain is a second reporter domain;
the fourth labeled nucleic acid domain is a second quencher domain; and
the second reporter domain and the second quencher domain are linked by the blocker nucleic acid domain.

53. The mixture of claim 51, wherein each of the plurality of hybridizing oligonucleotides further comprises a fourth labeled nucleic acid domain comprising a fourth label;
the third label and the fourth label are two spectrally similar or identical reporters.

54. The mixture of claim 51, wherein the third labeled nucleic acid domain is a third reporter domain; and
each of the plurality of hybridizing oligonucleotides comprises a quenching nucleotide that quenches an energy from the third label in an excited state.

55. A kit for quantitating a plurality of nucleic acid molecules comprising a plurality of target nucleic acid fragments, wherein each of the plurality of target nucleic acid fragments comprises a first adapter sequence, a target insert sequence, and a second adapter sequence; the first adapter sequence is linked to the second adapter sequence through the target insert sequence;
wherein the kit comprises:
a plurality of detectably-labeled probes;
a plurality of extension primers; and
instructions directing a user to (1) producing an extension product of each of the plurality of target nucleic acid fragments by extending a respective one of the plurality of extension primers with a polymerase; and (2) calculating a number of the plurality of target nucleic acid fragments based on signals detected upon a single cycle of extension reactions;
wherein each of the plurality of detectably-labeled probes comprises a first labeled nucleic acid domain comprising a first label;
at least a portion of each of plurality of the detectably-labeled probes is complementary to at least a portion of the first adapter sequence; and
at least a portion of each of the plurality of extension primers is complementary to at least a portion of the second adapter sequence.

56. The kit of claim 55, wherein each of the plurality of detectably-labeled probes further comprises a second labeled nucleic acid domain comprising a second label;
the first labeled nucleic acid domain is a first reporter domain; and
the second labeled nucleic acid domain is a first quencher domain.

57. The kit of claim 55, wherein each of the plurality of detectably-labeled probes further comprises a second labeled nucleic acid domain comprising a second label;
the first label and the second label are two spectrally similar or identical reporters.

58. The kit of claim 55, wherein the first labeled nucleic acid domain is a first reporter domain; and
each of the plurality of detectably-labeled probes comprises a quenching nucleotide that quenches an energy from the first label in an excited state.

59. The kit of claim 55, wherein the first adapter sequence comprises a first flow cell binding sequence;
at least a portion of each of the plurality of detectably-labeled probes is complementary to at least a portion of the first flow cell binding sequence.

60. The kit of claim 55, wherein the second adapter sequence comprises a second flow cell binding sequence; and
at least a portion of each of the plurality of extension primers is complementary to at least a portion of the second flow cell binding sequence.

61. The kit of claim 55, the kit further comprising a plurality of hybridizing oligonucleotides;
wherein each of the plurality of hybridizing oligonucleotides comprises a blocker nucleic acid domain.

62. The kit of claim 61, wherein each of the plurality of hybridizing oligonucleotides further comprises a third labeled nucleic acid domain comprising a third label.

63. The kit of claim 62, wherein each of the plurality of hybridizing oligonucleotides further comprises a fourth labeled nucleic acid domain comprising a fourth label;
the third labeled nucleic acid domain is a second reporter domain;
the fourth labeled nucleic acid domain is a second quencher domain; and
the second reporter domain and the second quencher domain are linked by the blocker nucleic acid domain.

64. The kit of claim 62, wherein each of the plurality of hybridizing oligonucleotides further comprises a fourth labeled nucleic acid domain comprising a fourth label;
the third label and the fourth label are two spectrally similar or identical reporters.

65. The kit of claim 62, wherein the third labeled nucleic acid domain is a third reporter domain; and
each of the plurality of hybridizing oligonucleotides comprises a quenching nucleotide that quenches an energy from the third label in an excited state.

66. The kit of any one of claims 55 to 65, the kit further comprising a plurality of nucleic acid binding dye molecules.

67. The kit of any one of claims 55 to 66, the kit further comprising a polymerase.


SEQUENCE LISTING (37 sequences)

SEQ ID NO: 1 - DNA, 20 bp, Artificial Sequence
  Description of Artificial Sequence Synthetic oligonucleotide
aatgatacggcgaccaccga

SEQ ID NO: 2 - DNA, 29 bp, Artificial Sequence
  Description of Artificial Sequence Synthetic oligonucleotide
aatgatacggcgaccaccgagatctacac

SEQ ID NO: 3 - DNA, 21 bp, Artificial Sequence
  Description of Artificial Sequence Synthetic oligonucleotide
tcgtatgccgtcttctgcttg

SEQ ID NO: 4 - DNA, 24 bp, Artificial Sequence
  Description of Artificial Sequence Synthetic oligonucleotide
atctcgtatgccgtcttctgcttg

SEQ ID NO: 5 - DNA, 33 bp, Artificial Sequence
  Description of Artificial Sequence Synthetic oligonucleotide
acactctttccctacacgacgctcttccgatct

SEQ ID NO: 6 - DNA, 34 bp, Artificial Sequence
  Description of Artificial Sequence Synthetic oligonucleotide
gtgactggagttcagacgtgtgctcttccgatct

SEQ ID NO: 7 - DNA, 33 bp, Artificial Sequence
  Description of Artificial Sequence Synthetic oligonucleotide
gatcggaagagcacacgtctgaactccagtcac

SEQ ID NO: 8 - DNA, 29 bp, Artificial Sequence
  Description of Artificial Sequence Synthetic oligonucleotide
gtgtagatctcggtggtcgccgtatcatt

SEQ ID NO: 9 - DNA, 29 bp, Artificial Sequence
  Description of Artificial Sequence Synthetic oligonucleotide
  Description of Combined DNA/RNA Molecule Synthetic oligonucleotide
  modified_base (10)..(10): 5-hydroxybutynl-2′-deoxyuridine
gtgtagatcncggtggtcgccgtatcatt

SEQ ID NO: 10 - DNA, 29 bp, Artificial Sequence
  Description of Artificial Sequence Synthetic oligonucleotide
  Description of Combined DNA/RNA Molecule Synthetic oligonucleotide
  modified_base (10)..(10): 5-hydroxybutynl-2′-deoxyuridine
  modified_base (17)..(17): 5-hydroxybutynl-2′-deoxyuridine
gtgtagatcncggtggncgccgtatcatt

SEQ ID NO: 11 - DNA, 19 bp, Artificial Sequence
  Description of Artificial Sequence Synthetic oligonucleotide
cggtggtcgccgtatcatt

SEQ ID NO: 12 - DNA, 35 bp, Artificial Sequence
  Description of Artificial Sequence Synthetic oligonucleotide
tgtagatctcggtggtcgccgtatcattatctaca

SEQ ID NO: 13 - DNA, 28 bp, Artificial Sequence
  Description of Artificial Sequence Synthetic oligonucleotide
tgtagatctcggtggtcgccgtatcatt

SEQ ID NO: 14 - DNA, 26 bp, Artificial Sequence
  Description of Artificial Sequence Synthetic oligonucleotide
cggtggtcgccgtatcattatctaca

SEQ ID NO: 15 - DNA, 36 bp, Artificial Sequence
  Description of Artificial Sequence Synthetic oligonucleotide
aactccagtcacatctcgtatgccgtcttctgcttg

SEQ ID NO: 16 - DNA, 31 bp, Artificial Sequence
  Description of Artificial Sequence Synthetic oligonucleotide
cagtcacatctcgtatgccgtcttctgcttg

SEQ ID NO: 17 - DNA, 24 bp, Artificial Sequence
  Description of Artificial Sequence Synthetic oligonucleotide
atctcgtatgccgtcttctgcttg

SEQ ID NO: 18 - DNA, 21 bp, Artificial Sequence
  Description of Artificial Sequence Synthetic oligonucleotide
ctcgtatgccgtcttctgctt

SEQ ID NO: 19 - DNA, 16 bp, Artificial Sequence
  Description of Artificial Sequence Synthetic oligonucleotide
gtatgccgtcttctgc

SEQ ID NO: 20 - DNA, 24 bp, Artificial Sequence
  Description of Artificial Sequence Synthetic oligonucleotide
caagcagaagacggcatacgagat

SEQ ID NO: 21 - DNA, 22 bp, Artificial Sequence
  Description of Artificial Sequence Synthetic oligonucleotide
caagcagaagacggcatacgag

SEQ ID NO: 22 - DNA, 20 bp, Artificial Sequence
  Description of Artificial Sequence Synthetic oligonucleotide
aagcagaagacggcatacga

SEQ ID NO: 23 - DNA, 19 bp, Artificial Sequence
  Description of Artificial Sequence Synthetic oligonucleotide
caagcagaagacggcatac

SEQ ID NO: 24 - DNA, 25 bp, Artificial Sequence
  Description of Artificial Sequence Synthetic oligonucleotide
aatgatacggcgaccaccgagatct

SEQ ID NO: 25 - DNA, 22 bp, Artificial Sequence
  Description of Artificial Sequence Synthetic oligonucleotide
gatacggcgaccaccgagatct

SEQ ID NO: 26 - DNA, 18 bp, Artificial Sequence
  Description of Artificial Sequence Synthetic oligonucleotide
gatacggcgaccaccgag

SEQ ID NO: 27 - DNA, 31 bp, Artificial Sequence
  Description of Artificial Sequence Synthetic oligonucleotide
acgctcttccgatctagatcggaagagcaca

SEQ ID NO: 28 - DNA, 28 bp, Artificial Sequence
  Description of Artificial Sequence Synthetic oligonucleotide
cgctcttccgatctagatcggaagagca

SEQ ID NO: 29 - DNA, 28 bp, Artificial Sequence
  Description of Artificial Sequence Synthetic oligonucleotide
cgctcttccgatctagatcggaagagca

SEQ ID NO: 30 - DNA, 35 bp, Artificial Sequence
  Description of Artificial Sequence Synthetic oligonucleotide
cgacgctcttccgatctagatcggaagagcacacg

SEQ ID NO: 31 - DNA, 35 bp, Artificial Sequence
  Description of Artificial Sequence Synthetic oligonucleotide
cgacggtcttccgatctagatccgaagagcacacg

SEQ ID NO: 32 - DNA, 120 bp, Artificial Sequence
  Description of Artificial Sequence Synthetic polynucleotide
aatgatacggcgaccaccgagatctacacacactctttccctacacgacgctcttccgatctagatcggaagagcacacgtctgaactccagtcacatctcgtatgccgtcttctgcttg

SEQ ID NO: 33 - DNA, 80 bp, Artificial Sequence
  Description of Artificial Sequence Synthetic oligonucleotide
aatgatacggcgaccaccgagatctacacacactctttcccttgaactccagtcacatctcgtatgccgtcttctgcttg

SEQ ID NO: 34 - DNA, 150 bp, Artificial Sequence
  Description of Artificial Sequence Synthetic polynucleotide
  modified_base (63)..(92): a, c, t, g, unknown or other
aatgatacggcgaccaccgagatctacacacactctttccctacacgacgctcttccgatctnnnnnnnnnnnnnnnnnnnnnnnnnnnnnnagatcggaagagcacacgtctgaactccagtcacatctcgtatgccgtcttctgcttg

SEQ ID NO: 35 - DNA, 116 bp, Artificial Sequence
  Description of Artificial Sequence Synthetic polynucleotide
gttcgtcttctgccgtatgctctacactgacctcaagtctgcacacgagaaggctagatctagccttctcgcagcacatccctttctcacatctagagccaccagcggcatagtaa

SEQ ID NO: 36 - DNA, 106 bp, Artificial Sequence
  Description of Artificial Sequence Synthetic polynucleotide
gttcgtcttctgccgtatgctctacactgacctcaagtctgcacacgatctagccttctcgcagcacatccctttctcacatctagagccaccagcggcatagtaa

SEQ ID NO: 37 - DNA, 86 bp, Artificial Sequence
  Description of Artificial Sequence Synthetic oligonucleotide
gttcgtcttctgccgtatgctctacactgacctcaagtctgcagcacatccctttctcacatctagagccaccagcggcatagtaa